(19) United States
(12) Patent Application Publication
(10) Pub. No.: US 2019/0133207 A1
(43) Pub. Date: May 9, 2019

(54) GARMENTS

(71) Applicant: Meer Group, LLC, Austin, TX (US)

(72) Inventors: Matthew DeIulio, Austin, TX (US); Arispe Adriana, Austin, TX (US)

(21) Appl. No.: 16/300,035

(22) PCT Filed: May 9, 2017
(86) PCT No.: PCT/US17/31816

§ 371 Date: Nov. 8, 2018

Related U.S. Application Data
(60) Provisional application No. 62/432,256, filed on Dec. 9, 2016.
(60) Provisional application No. 62/354,104, filed on Jun. 23, 2016.
(60) Provisional application No. 62/333,779, filed on May 9, 2016.

(51) Int. Cl. A41C 3/00; A41F 15/00; A41F 15/02; A61F 5/02

(52) U.S. Cl. CPC A41C 3/0028; A61F 5/026; A41F 15/02; A41F 15/002

(57) ABSTRACT

A garment comprising a support structure adapted to support breasts of a wearer; a first engagement feature coupled to an outer edge of the support structure on a first lateral side of the wearer and adapted to extend to a shoulder on a second lateral side of the wearer; a second engagement feature coupled to an outer edge of the support structure on a second lateral side of the wearer and adapted to extend to a shoulder on a first lateral side of the wearer, wherein, as viewed in-use, a vertical line extending along a back of the wearer intersects only the first and second engagement features, and wherein the first and second engagement features lie along best fit lines that intersect one another.

### FIELD OF THE DISCLOSURE

The present disclosure relates to garments.

### RELATED ART

Correct posture can enhance a person's overall well-being while facilitating improved quality of life. Correct posture can include alignment of the hips, spine, shoulders, neck, head, and other parts of the body with respect to one another to permit proper balance and biomechanical loading.

For some, correct posture is easily obtained through flexibility, existing muscle tone, and anatomical arrangement. For others, correct posture is difficult to obtain, worsened by improper postural alignment, underlying medical conditions, and the like. Traditional methods of breast support have proven unsatisfactory for proper biomechanical load distribution.

Postural improvement can lead to better quality of life, improved muscular tone and performance, improved range of motion and stability, and overall better well-being and enhanced quality of life. Consumers continue to demand garments capable of affecting postural improvement.

### BRIEF DESCRIPTION OF THE DRAWINGS

Embodiments are illustrated by way of example and are not limited by the accompanying figures.

FIG. 1 includes a perspective view of a garment in accordance with an embodiment.

FIG. 2 includes a rear view of the garment in accordance with an embodiment.

FIG. 3 includes a rear view of a garment in accordance with an embodiment.

FIG. 4 includes a side view of a garment in accordance with an embodiment.

FIG. 5 includes a rear view of a garment in accordance with an embodiment.

FIG. 6 includes a perspective view of a garment in accordance with an embodiment.

FIG. 7 includes a rear view of a garment in accordance with an embodiment.

FIG. 8 includes a perspective view of a garment in accordance with an embodiment.

FIG. 9 includes a rear view of a garment in accordance with an embodiment.

FIG. 10 includes a rear view of a garment in accordance with an embodiment.

FIG. 11 includes an enlarged elevation view of a junction in a garment in accordance with an embodiment.

FIG. 12 includes a rear view of a garment in accordance with an embodiment.

FIG. 13 includes a rear view of garments in accordance with an embodiment.

FIG. 14 includes a rear view of a garment in accordance with an embodiment.

FIG. 15 includes a rear view of a garment in accordance with an embodiment.

FIG. 16 includes a rear view of a garment in accordance with an embodiment.

Skilled artisans appreciate that elements in the figures are illustrated for simplicity and clarity and have not necessarily been drawn to scale. For example, the dimensions of some of the elements in the figures may be exaggerated relative to other elements to help to improve understanding of embodiments of the invention. Embodiments illustrated in separate figures may be provided in combination in a single embodiment, and conversely, various features that are, for brevity, illustrated in the context of a single embodiment, may also be provided separately or in any subcombination.

### DETAILED DESCRIPTION

The following description in combination with the figures is provided to assist in understanding the teachings disclosed herein. The following description will focus on specific implementations and embodiments of the teachings. This focus is provided to assist in describing the teachings and should not be interpreted as a limitation on the scope or applicability of the teachings.

As used herein, the terms “comprises,” “comprising,” “includes,” “including,” “has,” “having,” or any other variation thereof, are intended to cover a non-exclusion inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

The use of “a” or “an” is employed to describe elements and components described herein. This is done merely for convenience and to give a general sense of the scope of the invention. This description should be read to include one or at least one and the singular also includes the plurality, or vice versa, unless it is clear that it is meant otherwise. Unless otherwise defined or conflicting, the terms “vertical,” “horizontal,” “lateral,” and the like are intended to refer to directional orientations as they relate to the orientations illustrated in the figures. Unless otherwise defined, the terms “in use,” “in-use,” “engaged,” or the like are intended to refer to a condition where the garment is at least partially disposed on a body, mannequin, form, shape resembling a human, or the like. The terms “not-in-use,” “out of use,” “not engaged,” “disengaged,” and the like are intended to refer to a condition where the garment is not disposed on a body, mannequin, form, shape resembling a human, or the like. Where not conflicting with other disclosure herein and not otherwise defined, values described using terms “generally,” “substantially,” and the like are intended to refer to a value ±10%, or ±8%, or ±5%, or ±3%, or ±1% the stated value as understood by one of ordinary skill in the art.

Unless otherwise defined, all technical and scientific terms used herein have the same meaning as commonly understood by one of ordinary skill in the art to which this invention belongs. Unless otherwise defined and where not conflicting, the terms “shoulder” and “arm” can be used interchangeably. Further, where appropriate as understood by one of ordinary skill, reference to a specific lateral side of the body or any portion associated therewith may be interchangeable with the other lateral side of the body or any portion associated therewith. The materials, methods, and examples are illustrative only and not intended to be limiting. To the extent not described herein, many details regarding specific materials and processing acts are conventional and may be found in textbooks and other sources within the garment and textile arts.

Garments in accordance with embodiments described herein are generally adapted to be worn on the body. Garments in accordance with certain embodiments described herein can reduce back pain, shoulder pain, neck pain, or other bodily pains. Garments in accordance with some embodiments may increase wearer flexibility and strength. Garments in accordance with particular embodiments may increase postural alignment of the body.

Garments described in accordance with particular embodiments herein are adapted to be worn at least partially on the torso of the body and can extend across portions of the chest and back. Without wishing to be restricted to the following, the garment can be, or be a part of, a brassiere (bra), swimwear, shirt, sweater, jacket, vest, other clothing article, or any combination thereof. In an embodiment, the garment is adapted to be slipped on and off the body over the shoulders. In a more particular embodiment, the garment can be devoid of selective clasps which permit at least partial opening of the garment. In another embodiment, the garment can be at least partially opened and closed, for example with a clasp, zipper, button, or other readily known attachment mechanism recognized in the art by one or ordinary skill. In a particular embodiment, the garment can be opened and closed from the front. In another embodiment, the garment can be opened and closed from the back. In yet a further embodiment, the garment can be opened and closed from one or both sides. In an embodiment, the garment is reversible. That is, the garment can be worn inside-out.

In an embodiment, the garment includes a support structure and an engagement structure. The support structure can include a portion of the garment adapted to be disposed generally along the chest of the body. The engagement structure can include a portion of the garment adapted to be disposed generally along the back of the body. In certain embodiments the support structure can extend at least partially to the back of the body. In certain embodiments the engagement structure can extend at least partially to the front of the body. In a particular embodiment, at least a portion of the support structure and engagement structure have a unitary construction. In another particular embodiment, the support structure and engagement structure can overlap, intersect, join together at selected locations, or otherwise occupy common locations of the garment.

In an embodiment, the garment includes a portion, such as a band (e.g., a chest band), extending from the chest of the body toward the back of the body. The band can be unitary with the engagement structure or support structure and can be defined by the portion of the garment under a lower edge of cups of the support structure. In an embodiment, the band and support structure are continuously, or substantially continuously, coupled together along the chest of the wearer's body. The engagement structure can include a portion extending at least partially behind the back of the body between opposite lateral sides of the body. In a particular embodiment, the engagement structure can include a first engagement feature and a second engagement feature. In a particular instance, the first engagement feature can be coupled with the band at a first lateral sides thereof and the second engagement feature can be coupled with the band at a second, opposite lateral side thereof.

The first engagement feature can be coupled to the second lateral side shoulder while the second engagement feature can be coupled to the first lateral side shoulder. In such a manner, the first and second lateral sides of the support structure can be coupled to the opposite shoulder of the body via a rear connection. In an embodiment, application of force through the first and second engagement features can reduce perceived weight of the wearer's breasts. In another embodiment, the garment can selectively bias the shoulders into a posture enhancing position.

In an embodiment, force applied to the shoulders can include a downward force, or a rearward force, or an inward force, or any combination thereof. As used herein, “downward force” is a force having a vector extending generally down toward the ground when the wearer is standing upright with the garment engaged with the body. Downward force can be oriented generally parallel with the spine. As used herein, “rearward force” is a force having a vector extending generally rearward away from the back of the body. Rearward force can be oriented generally parallel with the ground, or perpendicular to the spine. As used herein, “inward force” is a force applied toward an opposite lateral side of the body. For example, an inward force applied to the right shoulder has a force vector generally oriented toward the left shoulder. Similarly, inward force applied to the left shoulder has a force vector generally oriented toward the right shoulder. Downward, rearward, or inward forces can act independently or in concert in any combination to generate a postural enhancing effect, repositioning portions of the torso, such as the shoulders, to a more desirable location relative to the spine. In an embodiment, the garment is adapted to provide downward force in combination with at least one of inward and rearward force to at least one of the shoulders. In another embodiment, the garment is adapted to provide inward force in combination with at least one of downward force and rearward force to at least one of the shoulders. In yet a further embodiment, the garment is adapted to provide rearward force in combination with at least one of downward force and inward force to at least one of the shoulders.

In a particular embodiment, the garment is adapted to provide a force, F, to at least one shoulder of the body. In an embodiment, the force, F, includes a rearward component, F_{R}, a downward component, F_{D}, and an inward component, F_{I}. In a particular instance, F_{R}≥F_{D}≥F_{I}. In another embodiment, F_{R}≥F_{I}≥F_{D}. In yet another embodiment, F_{D}≥F_{R}≥F_{I}. In a further embodiment, F_{D}≥F_{I}≥F_{R}. In yet a further embodiment, F_{I}≥F_{R}≥F_{D}. In yet another embodiment, F_{I}≥F_{D}≥F_{R}. In an embodiment, the relative magnitude of each of the three vectors can be selectively changed by the wearer. In other embodiments described herein, the relative magnitudes of each of the three vectors can be generally fixed with respect to one another.

In an embodiment F_{D} is at least 0.01 F_{I}, or at least 0.05 F_{I}, or at least 0.1 F_{I}, or at least 0.2 F_{I}, or at least 0.3 F_{I}, or at least 0.4 F_{I}, or at least 0.5 F_{I}, or at least 0.75 F_{I}, or at least 1.0 F_{L} In another embodiment, F_{D} is no greater than 100 F_{I}, or no greater than 50 F_{I}, or no greater than 10 F_{I}, or no greater than 5 F_{I}. In a further embodiment, F_{D} is at least 0.01 F_{R}, or at least 0.05 F_{R}, or at least 0.2 F_{R}, or at least 0.3 F_{R}, or at least 0.4 F_{R}, or at least 0.5 F_{R}, or at least 0.75 F_{R}, or at least 1.0 F_{R}. In another embodiment, F_{D} is no greater than 100 F_{R}, or no greater than 50 F_{R}, or no greater than 10 F_{R}, or no greater than 5 F_{R}. In an embodiment, F_{I} is at least 0.091 F_{R}, or at least 0.05 F_{R}, or at least 0.1 F_{R}, or at least 0.2 F_{R}, or at least 0.3 F_{R}, or at least 0.4 F_{R}, or at least 0.5 F_{R}, or at least 0.75 F_{R}, or at least 1.0 F_{R}. In another embodiment, F_{I} is no greater than 100 F_{R}, or no greater than 50 F_{R}, or no greater than 10 F_{R}, or no greater than 5 F_{R}.

In an embodiment, force applied by the garment (such as for example by the first or second engagement features on the opposite shoulder) is adapted to be adjustable. That is, a wearer can adjust the force applied by the garment on the body, thus allowing the wearer to readily select appropriate support, breast lift, or posture enhancement as desired. In a particular embodiment, the garment can define an operable length, as measured by the combined lengths of the first engagement feature, the second engagement feature, and the support structure coupling the first and second engagement features together. The operable length can be measured by the shortest distance between opposite arm engagement portions as measured through the engagement features and the support structure. In an embodiment, the operable length is adjustable. Adjusting the operable length of the garment allows the wearer to adjust forces applied to the body (e.g., the shoulders). In an embodiment, adjustment of the operable length can occur along the first engagement feature. For example, the first engagement feature can have an adjustable element which permits resizing of the first engagement feature. In another embodiment, adjustment of the operable length can occur along the first and second engagement features. In yet another embodiment, adjustment of the operable length can occur along the support structure. For example, the band can be adjustable in length or the support structure can include an element which shortens the distance between the engagement features. In a further embodiment, the operable length can be adjusted by any combination of the first engagement feature, the second engagement feature, and the support structure. Traditional garments avoid such adjustability as it was believed to reduce support to breast tissue, however, garments in accordance with embodiments described herein are adapted to provide more than adequate support and lift to breast tissue even when the operable length is made larger (i.e., the garment is loosened).

In an embodiment, a wearer can put on the garment and subsequently tighten the operable length after positioning the garment on the body. Reducing the operable length of the garment once the garment is on the body can provide posture enhancing support to the shoulders and breast tissue of the body by redirecting traditional downward force along the shoulders in a more circumferential, rearward direction. The operable length can be infinitely adjustable such that the wearer can adjust the operable length to any desired length between a minimum and maximum length thereof. That is, the operable length can be defined by limitless adjustment locations instead of fixed points which would only permit limited operable lengths. In an embodiment, the garment can include fixed points for adjustment if desired. It is noted that the use of a below-shoulder portion (i.e., the portion of the arm engagement portion that extends below the shoulder of the wearer) can create enhanced breast support as compared to traditional bras as the force applied by the engagement feature on the below-shoulder portion of the arm engagement portion provides upward and inward support to breast tissue.

In an embodiment, the garment is selectively adjustable between a first configuration and a second configuration. In the first configuration the first and second engagement features can form an “X” on the back of the body. More particularly, the first and second engagement features can lie along best fit lines that intersect one another. In the second configuration the first and second engagement features can be generally parallel with respect to one another or lie along best fit lines that are generally parallel with respect to one another. In a particular instance, adjustment between the first and second configurations can allow a wearer to selectively engage and disengage posture-enhancing forces on the shoulders of the wearer's body.

In an embodiment, the garment is adapted such that only the first and second engagement features cross behind (over) the spine of the back of the body. The back of the garment can be devoid of a horizontal back band such as those found along the back of conventional bras. In a particular embodiment, the garment is adapted such that a vertical line extending along the back of a wearer only intersects the first and second engagement features. In a more particular embodiment, the vertical line extending along the back of the wearer that only intersects the first and second engagement features can be parallel with and extend over the spine of the wearer. In another more particular embodiment, the vertical line can be displaced laterally from the spine of the wearer (i.e., offset from the center of the back).

In an embodiment, in-use at least a portion of the circumference of the garment can include only the first and second engagement features. For example, at least 5% of the circumference of the wearer can include only the first and second engagement features, or at least 10% of the circumference of the wearer can include only the first and second engagement features, or at least 20% of the circumference of the wearer can include only the first and second engagement features, or at least 30% of the circumference of the wearer can include only the first and second engagement features, or at least 40% of the circumference of the wearer can include only the first and second engagement features. In an embodiment, the portion of the garment including only the first and second engagement features can be adapted to be disposed along the back of the wearer. In a more particular embodiment, the portion of the garment including only the first and second engagement features can be centered, or generally centered, relative to the back of the wearer.

In a particular embodiment, the portion of the circumference of the garment which includes only the first and second engagement features can include secondary elements such as sleeves, hooks, rings, fabric, webbing, attachments (e.g., indicia), or any combination thereof which nominally affects force transfer of the engagement structure on the shoulders of the wearer. In another embodiment, the first and second engagement features are freely moveable with respect to one another within the portion of the circumference including only the first and second engagement features. For example, the first and second engagement features can be detached from one another or secured together by any secondary means which does not restrict relative movement between the first and second engagement features.

In an embodiment, at least one of the first and second engagement features can be statically coupled with the first or second lateral sides of the support structure. In another embodiment, at least one of the first and second engagement features can be dynamically coupled with the first or second lateral sides of the support structure. Dynamic engagement can include, for example, repositionable engagement; detachable engagement; or other adjustable, non-static engagement.

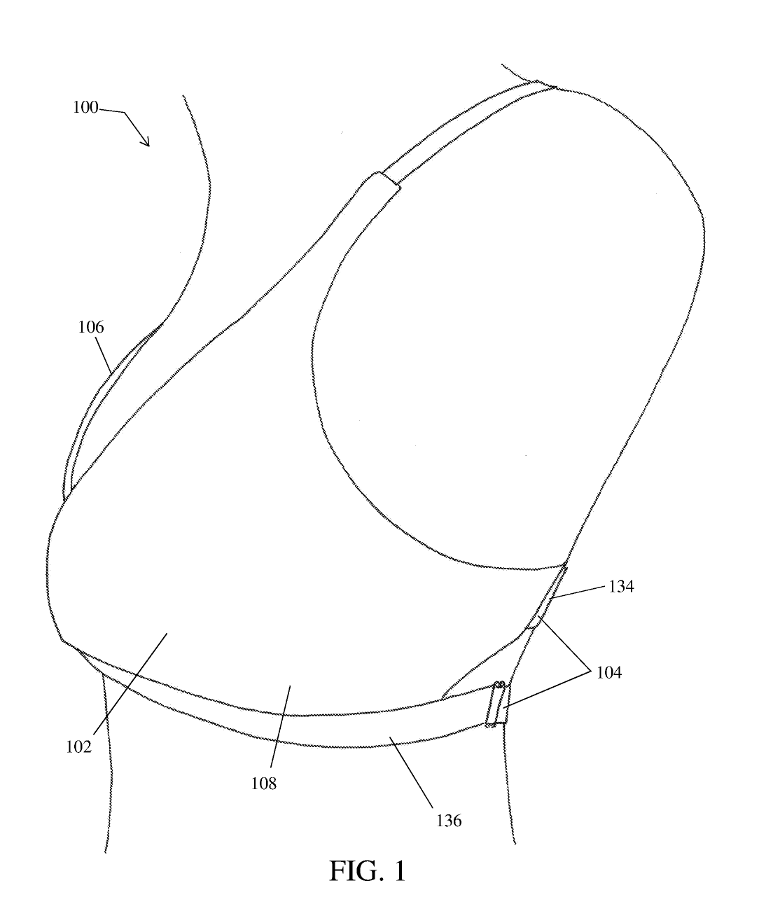

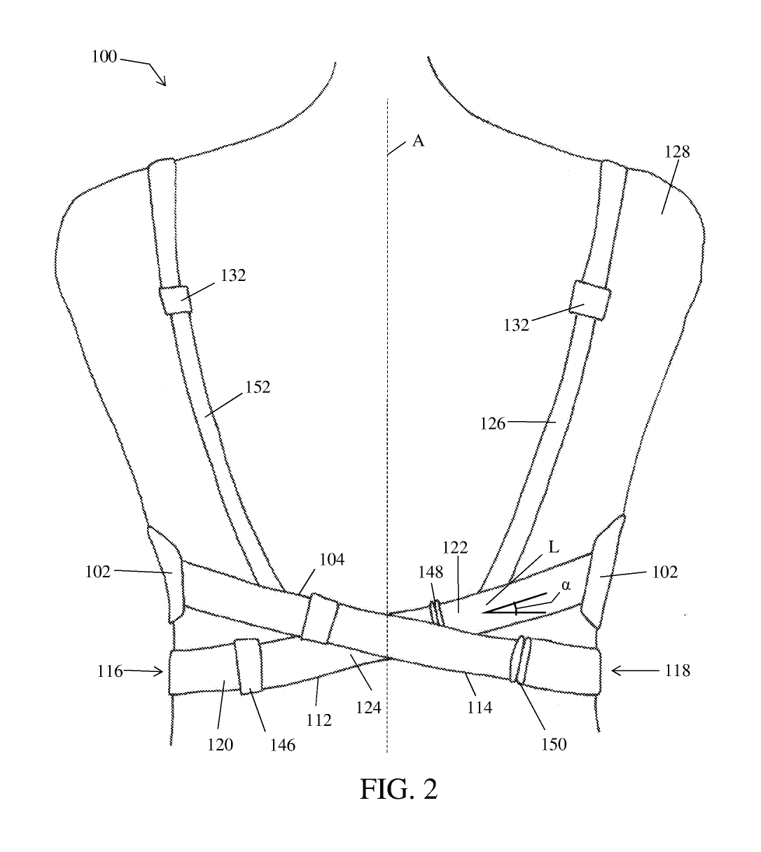

Referring to the figures, FIG. 1 includes a front perspective view of a garment 100 in accordance with an embodiment. FIG. 2 includes a rear view of the garment 100 in accordance with an embodiment. The garment 100 in FIGS. 1 and 2 is illustrated in an in-use configuration as it may appear when worn on a body or form.

In an embodiment, the garment 100 generally includes a support structure 102 and an engagement structure 104 coupled to the support structure 102. The support structure 102 includes a first cup 106 and a second cup 108. In certain embodiments the garment 100 can include a non-discriminatory cup volume (e.g., compression sports bras) where use of terms “first cup” and “second cup” are intended to refer to first and second portions of the non-discriminatory cup volume.

In an embodiment, the support structure 102 can be devoid of an underwire. In another embodiment, the support structure 102 can be essentially free of an underwire. It is noted that the support and vertical lift provided by the garment 100 on breasts of wearers has been observed to be on par and even better than that of garments (e.g., bras) including underwire. Thus, wearers can achieve great support and vertical lift using the garment 100 without compromising comfort.

In an embodiment, at least one of the first and second cups 106 and 108 can be detachably engaged with the remaining support structure 102. The garment 100 can be worn while nursing an infant without requiring complete removal of the garment 100. In an embodiment, the first and second cups 106 and 108 can be detachably coupled together (e.g., embodiments where the garment 100 is opened and closed from the front).

In an embodiment, the garment 100 can include a band 136 extending from the front of the body toward the back of the body. In an embodiment, the band 136 can have a unitary construction with the engagement structure 104. In another embodiment, the band 136 can have a unitary construction with the support structure 102. In a further embodiment, the band 136 and at least one of the cups 106 or 108 include separate, discrete pieces coupled together. In the illustrated embodiment, the band 136 is disposed adjacent to at least one of the cups 106 or 108 without any gaps between the cups 106 or 108 and the band 136. In an embodiment, one or more gaps can exist between at least one of the cups 106 or 108 and the band 136.

FIG. 2 illustrates an embodiment of the engagement structure 104, including a first engagement feature 112 and a second engagement feature 114. The first engagement feature 112 can be coupled with a first lateral side 116 of the support structure 102 and the second engagement feature 114 can be coupled with a second lateral side 118 of the support structure 102. In an embodiment, at least one of the first and second engagement features 112 and 114 is statically coupled to the support structure 102 in a non-adjustable configuration. In another embodiment, as illustrated for example in FIG. 8, and described below in greater detail, at least one of the first and second engagement features can be dynamically (e.g., adjustably or detachably) coupled to the support structure 102. By way of non-limiting examples, dynamic engagement can permit rotational, translational, or longitudinal movement; tightening; repositioning; detaching; or any combination thereof of the first or second engagement feature 112 or 114 with respect to the support structure 102.

While the following description is made with respect to the first engagement feature 112, it should be understood that the second engagement feature 114 can include any number of similar or different attributes as described with respect to the first engagement feature 112. In an embodiment, the first and second engagement features 112 and 114 are adapted to be reflectively, or generally reflectively, symmetrical about a vertical axis, e.g., the spine. In another embodiment, the first engagement feature 112 can have one or more different properties as compared to the second engagement feature 114. For example, the first and second engagement features 112 and 114 can be asymmetrical. In a particular embodiment, asymmetrical engagement features 112 and 114 may be suitable for wearers spending significant time in non-symmetric body positions, such as for example, sitting at a desk, driving, performing certain athletic activities, or sleeping. Asymmetrical engagement features 112 and 114 may also provide compensation for asymmetrical body shapes, such as asymmetrical breasts. In an embodiment, force from the garment 100 can be transmitted to only one shoulder of the wearer while force is omitted, or generally omitted, from the other shoulder. In another embodiment, the force adapted to be transmitted to a first shoulder by the garment 100 can be different than the force adapted to be transmitted to a second shoulder by the garment 100. Different forces can provide compensation for asymmetrical body shapes, such as asymmetrical breasts. It is noted that the adjustability of the garment 100 can permit a wearer to use a symmetrical garment in an asymmetrical configuration (e.g., loosening one of the engagement features 112 or 114 relative to the other engagement feature 112 or 114).

In an embodiment, the first engagement feature 112 can have a first end 120 extending toward the first lateral side 116 of the support structure 102 and a second end 122 extending toward an opposite shoulder disposed at the second lateral side 118 of the support structure 102. In an embodiment, the second end 122 can be coupled directly to the support structure 102. In another embodiment, the second end 122 can be spaced apart from the support structure 102 by an arm engagement portion (described below in greater detail). A middle portion 124 can be disposed between the first end 120 and second end 122. In an embodiment, the first and second ends 120 and 122 and the middle portion 124 of the first engagement feature 112 can all share the same dimensions (e.g., width and thickness). In another embodiment, at least two of the first end 120, second end 122, and middle portion 124 can have different dimensions as compared to one another. By way of a non-limiting embodiment, the middle portion 124 can be narrower than the first and second ends 120 and 122. In another embodiment, the second end 122 can be narrower than the first end 120. In yet a further embodiment, the first end 120 can be narrower than the second end 122. Different dimensions along the first engagement feature 112 may facilitate, for example, desired loading characteristics along the garment 100, reduction in weight and volume of the garment 100, more ergonomic feel of the garment, increased aesthetic, or any combination thereof. By way of another non-limiting embodiment, at least two of the first end 120, second end 122, and middle portion 124 can have different elastic moduli as compared to one another. For example, the first end 120 can have a higher elastic modulus than the middle portion 124 or second end 122. In another example, the second end 122 can have a lower elastic modulus than the first end 120.

As illustrated in FIG. 2, the first and second engagement features 112 and 114 can overlap one another. In an embodiment, the radial arrangement of the engagement feature 112 or 114 can be selectively switchable. That is, the wearer can put the first engagement feature 112 inside or outside of the second engagement feature 114. In a particular embodiment, the first and second engagement features 112 and 114 float relative to one another. As used herein, “float” refers to a condition whereby two or more components can move independent of one another (e.g., the two or more components are not statically affixed). In a particular embodiment, the first and second engagement features 112 and 114 float at the location where the first and second engagement features 112 and 114 overlap. In a particular instance, the first and second engagement features 112 and 114 are not connected together at the location of overlap.

In an embodiment, the first and second engagement features 112 and 114 can be coupled together via a coupling element (not illustrated). Such coupling may not be intended to prevent the first and second engagement features 112 and 114 from floating relative to one another. However, in a particular embodiment, the coupling element can couple the first and second engagement features 112 and 114 together at the location of overlap. In an embodiment, the coupling element is not fixedly secured to at least one of the first and second engagement features 112 and 114. This can allow the first and second engagement features 112 and 114 to move and deform relative to one another without affecting, or significantly affecting, each other. In a particular instance, the garment 100 can further include a centering element (not illustrated) adapted to maintain the coupling element at a central location with respect to the first and second engagement features 112 and 114 (e.g., near the spine of the body). The centering element can include fabric, bands, straps, or the like, which extend between the coupling element and another location along the garment.

In an embodiment, the garment 100 can include a stay (not illustrated) adapted to selectively secure the first or second engagement features 112 or 114 at a relatively fixed position with respect to one another. In this regard, the wearer can selectively adjust and secure the first or second engagement features 112 or 114 to each other, to the centering element, to another portion of the garment 100, or to any combination thereof.

In an embodiment, the first engagement feature 112 includes an adjustable element 146 adapted to adjust an attribute (such as length) of the first engagement feature 112. In a further embodiment, the first engagement feature 112 includes a plurality of adjustable elements (not illustrated) each adapted to adjust the attribute (e.g., length) of the first engagement feature 112 at different locations. The adjustable element 146 can be disposed at the first end 120 of the first engagement feature 112, or the second end 122 of the first engagement feature 112, or the middle portion 124 of the first engagement feature 112, or a combination thereof.

In an embodiment, the adjustable element 146 includes a slider (e.g., adjustable element 1224 illustrated in FIG. 12). In a particular embodiment, the adjustable element 146 can include a grasping element (not illustrated) coupled thereto and adapted to permit easier grasp of the adjustable element 146. The grasping element can include a pull tab, a lip, a groove, a textured surface, another suitable grasping arrangement, or any combination thereof. In an embodiment, the grasping element can include the same material as the underlying material of the first engagement feature 112. In another embodiment, the grasping element can include a different material as compared to the underlying material of the first engagement feature 112. In an embodiment, the grasping element can have a same width, as measured perpendicular to the length of the first engagement feature 112, as the underlying material of the first engagement feature 112. In another embodiment, the grasping element can have a width less than the width of the underlying material of the first engagement feature 112. In yet another embodiment, the grasping element can have a width greater than the width of the underlying material of the first engagement feature 112. In an embodiment, the grasping element can include a textured portion adapted to enhance grip between a user's hand and the grasping element. By way of non-limiting example, the grasping element can be disposed at least partially along an outer surface of the first engagement feature 112, at least partially along an inner surface of the first engagement feature 112, at least partially along a side surface of the first engagement feature 112, or any combination thereof. In an embodiment, the grasping element is connected to the remaining garment only at the adjustable element 146. In another embodiment, the grasping element can be coupled to the first engagement feature 112 so as to prevent the grasping element from freely swinging or pulling away from the first engagement feature 112. By way of non-limiting example, the grasping element can include a portion extending at least partially around the first engagement feature 112. The portion extending at least partially around the first engagement feature 112 can return or maintain the grasping element at a location generally proximate to the first engagement feature 112 when the user is not grasping the grasping element.

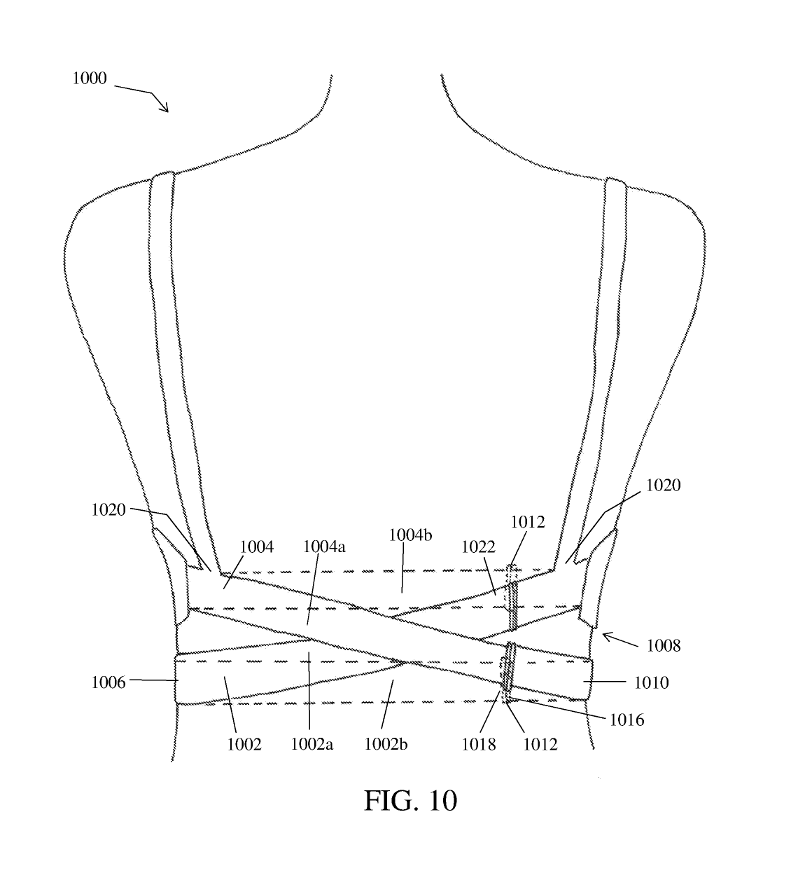

In an embodiment, the first engagement feature 112 (or the first arm engagement portion described below in greater detail) can include a disconnectable element 148, described below in greater detail, which allows a user to selectively detach a portion of the first engagement feature 112 (e.g., near end 122) from another portion of the first engagement feature 112 (e.g., middle portion 124). In an embodiment, the second engagement feature 114 or second arm engagement portion can include a disconnectable element 150. In an embodiment, the disconnectable elements 148 and 150 can be disposed at relatively similar lateral locations along the first and second engagement features 112 and 114. In another embodiment, the disconnectable elements 148 and 150 can be disposed at different lateral locations along the first and second engagement features 112 and 114. As described in greater detail with respect to FIG. 10, in an embodiment the disconnectable elements 148 and 150 can be interchangeable such that the garment 100 can be reconfigured between at least two different configurations.

In a particular embodiment, the garment 100 is adapted such that a vertical line A extending along the back of a wearer only intersects the first and second engagement features 112 and 114. The vertical line A can be disposed at a central location of the back of the wearer or displaced a lateral distance therefrom. In an embodiment, in-use at least a portion of the circumference of the garment 100 can include only the first and second engagement features 112 and 114. For example, at least 5% of the circumference of the wearer can include only the first and second engagement features 112 and 114, or at least 10% of the circumference of the wearer can include only the first and second engagement features 112 and 114, or at least 20% of the circumference of the wearer can include only the first and second engagement features 112 and 114, or at least 30% of the circumference of the wearer can include only the first and second engagement features 112 and 114, or at least 40% of the circumference of the wearer can include only the first and second engagement features 112 and 114. In an embodiment, the portion of the garment 100 including only the first and second engagement features 112 and 114 can be adapted to be disposed along the back of the wearer. In a more particular embodiment, the portion of the garment 100 including only the first and second engagement features 112 and 114 can be adapted to be centered, or generally centered, relative to the back of the wearer.

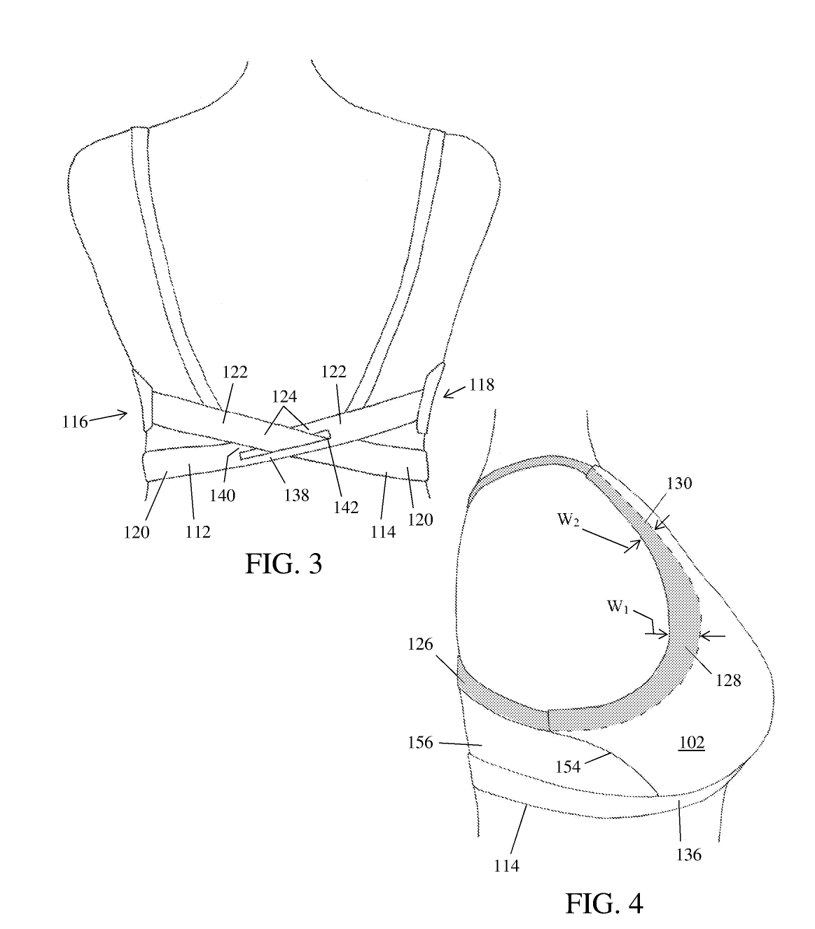

Referring to FIG. 3, in an embodiment, the first engagement feature 112 includes at least two sub-features 138 and 140 spaced apart by a slit 142. In an embodiment, the second engagement feature 114, or a portion thereof, can extend through the slit 142 of the first engagement feature 112 (e.g., over one sub-feature 140 and under another sub-feature 138). In a particular instance, such woven configuration can prevent the garment 100 from becoming tangled or undone such as, for example, when washed or taken off in embodiments where the garment 100 can be opened. In an embodiment, the slit 142 can include a plurality of slits,

In a particular embodiment, the number, Ni, of sub-features on the first engagement feature 112 can be the same as the number, N₂, of sub-features on the second engagement feature 114. In another embodiment, N₁ can be different than N₂. For example, a ratio [N₁/N₂] of sub-features can be in a range of 0.01 and 0.99, or in a range of 0.1 and 0.8, or in a range of 0.3 and 0.6, or in a range of 1.01 and 50.0, or in a range of 1.01 and 3.0.

In an embodiment, all sub-features 138 and 140 of the first engagement feature 112 are adapted to transmit force between the first and second lateral sides 116 and 118 of the body. In a particular embodiment, all of the sub-features 138 and 140 can be adapted to transmit equal, or generally equal, force. In another particular embodiment, at least one of the sub-features 138 or 140 can be adapted to transmit a different force as compared to the other sub-feature 138 or 140. In other instances, less than all of the sub-features 138 and 140 (N₁−n, where n is at least 1) can transmit force between the first and second lateral sides 116 and 118. For example, by way of a non-limiting example, the first engagement feature 112 can include three sub-features, only two of which transmit force to the second lateral side 118 shoulder. The third sub-feature can be aesthetic or can satisfy another purpose (e.g., connection of detachable components described below in greater detail or reconfiguration as described below in greater detail).

In an embodiment, the first engagement feature 112 can have at least three sub-features, or at least four sub-features, or at least five sub-features, or even at least ten sub-features. In another embodiment, the first engagement feature 112 can include no greater than fifty sub-features, or no greater than twenty sub-features, or no greater than ten sub-features. In an embodiment, at least two adjacent slits 142 have equal, or generally equal, lengths as compared to one another. In another embodiment, at least two adjacent slits 142 have different lengths as compared to one another.

In an embodiment, the length of at least one of the slits 142, as measured parallel with a length of the first engagement feature 112, is greater than the width of a portion of the second engagement feature 114 extending therethrough. For example, the length of the at least one slit 142 can be at least 101% the width of the portion of the second engagement feature 114 extending therethrough. In another embodiment, the length of the at least one slit 142 is not greater than 1,000% the width of the portion of the second engagement feature 114 extending therethrough. Use of a slit 142 with a greater length than the width of the corresponding portion of the engagement feature extending therethrough may prevent binding between the first and second engagement features 112 and 114 while permitting maximum flexure of the garment 100 to accommodate various body sizes and types as well as desired tension.

Referring again to FIG. 2, the garment 100 can further include a first arm engagement portion 126 coupled with the first engagement feature 112. The first arm engagement portion 126 is adapted to extend around at least a portion of the second lateral side 118 shoulder of the wearer. The first arm engagement portion 126 can transmit rearward force, or downward force, or inward force, or a combination of forces to the shoulder and support structure 102. In a particular instance, transmission of such forces can reduce occurrence of forward rounded shoulders or relieve postural imbalance caused (for example) by the weight of breast tissue.

The first arm engagement portion 126 is adapted to extend at least partially around the shoulder of the wearer. In an embodiment, the first arm engagement portion 126 is adapted to extend around at least 1% of the circumference of the shoulder, or at least 25% of the circumference of the shoulder, or at least 50% of the circumference of the shoulder, or at least 99% of the circumference of the shoulder. In a particular embodiment, the first arm engagement portion 126 is adapted to extend around the entire circumference (i.e., 100%) of the shoulder.

In an embodiment, the first arm engagement portion 126 defines an opening 128 through which the shoulder of the wearer can extend. The opening 128 can be arcuate or polygonal, such as triangular, rectangular, or pentagonal. In yet a further embodiment, the opening 128 includes arcuate and polygonal portions. In a particular embodiment, the opening 128 can have a generally tear-drop shape when in use. The apex of the tear drop can be disposed adjacent to the rear of the body. In an embodiment, the second end 122 of the first engagement feature 112 can be coupled with or near the apex of the tear drop shaped first arm engagement portion 126.

In an embodiment, the first arm engagement portion 126 is adjustable. Adjustability may permit accommodation of various sized and shaped shoulders. In an embodiment, the first arm engagement portion 126 includes one or more adjustment elements 132 adapted to resize or reshape the opening 128. In an embodiment, at least one of the adjustment elements 132 can be disposed in a hidden area of the garment 100. In another embodiment, at least one of the adjustment elements 132 can be at least partially, such as entirely, visible. In an embodiment, the first arm engagement portion 126 can include a disconnectable element (not illustrated). The disconnectable element can allow for selective opening of the garment 100 along the first arm engagement portion 126 or at an interface between the first arm engagement portion 126 and the first engagement feature 112. In an embodiment, the disconnectable element can be disposed at an elevation below the shoulder of the wearer. In another embodiment, the disconnectable element can be disposed at an elevation common with the shoulder of the wearer. In yet another embodiment, the disconnectable element can be disposed at an elevation above the shoulder of the wearer.

In the embodiment, the first arm engagement portion 126 can be coupled to the first engagement feature 112 at one location, L. In another, non-illustrated embodiment, the first arm engagement portion 126 can be coupled to the first engagement feature 112 at a plurality of locations, such as at least two locations, or at least three locations, or at least four locations. For example, at least two members can extend between the first engagement feature 112 and the first arm engagement portion 126. In a particular embodiment, at least one of the members extending between the first engagement feature 112 and first arm engagement portion 126 can be adjustable (e.g., adjustable in length). In another embodiment, all of the members extending between the first engagement feature 112 and the first arm engagement portion 126 can be adjustable. In a further embodiment, all of the members extending between the first engagement feature 112 and the first arm engagement portion 126 can be non-adjustable.

In an embodiment, the first arm engagement portion 126 and the first engagement feature 112 are coupled together along an inner-lower-rear portion of the first arm engagement portion 126 (e.g., near the shoulder blade of the wearer). In an embodiment, the coupling between the first arm engagement portion 126 and first engagement feature 112 is fixed. In another embodiment, the coupling between the first arm engagement portion 126 and the first engagement feature 112 is dynamic (e.g., adjustable). In another embodiment, the first arm engagement portion 126 and the first engagement feature 112 have an at least partially unitary construction. By way of a non-limiting example, the material of the first engagement feature 112 can extend to the support structure 102 and form at least a portion of the first arm engagement portion 126.

In an embodiment, the first arm engagement portion 126 can have a generally uniform width, as measured around the circumference of the shoulder. In another embodiment, illustrated in FIG. 4, the first arm engagement portion 126 can have a non-uniform width, as measured around the circumference of the shoulder. For example, a first region 128 of the first arm engagement portion 126 can have a first width, W₁, and a second region 130 of the first arm engagement portion 126 can have a second width, W₂, different from the first width, W₁. In an embodiment, the first width can be greater than the second width. For example, W₁ can be at least 1.01 W₂, or at least 1.05 W₂, or at least 1.2 W₂, or at least 1.5 W₂, or at least 2.0 W₂. In another embodiment, W₁ is no greater than 75.0 W₂. In an embodiment, the first region 128 can be disposed at or adjacent to a location where the garment 100 is adapted to transmit the greatest force to the shoulder.

In a particular instance, the first region 128 can extend around at least 1% of the circumference of the first arm engagement portion 126, or at least 5% of the circumference of the first arm engagement portion 126, or at least 20% of the circumference of the first arm engagement portion 126, or at least 50% of the circumference of the first arm engagement portion 126, or at least 75% of the circumference of the first arm engagement portion 126, or at least 99% of the circumference of the first arm engagement portion 126. In a further instance, the first region 128 extends around no greater than 99.9% of the circumference of the first arm engagement portion 126.

The first arm engagement portion 126 is illustrated with shading and dashed lines in FIG. 4. In an embodiment, the first arm engagement portion 126 can have a monolithic construction. In another embodiment, the first arm engagement portion 126 can have a multi-piece construction.

The first arm engagement portion 126 can be coupled with the support structure 102 at one or more locations. In a particular embodiment, at least a portion of the first arm engagement portion 126 is disposed within the support structure 102. In a more particular embodiment, at least 10% of the length of the first arm engagement portion 126 is disposed within the support structure 102, or at least 25% of the length of the first arm engagement portion 126 is disposed within the support structure 102, or at least 50% of the length of the first arm engagement portion 126 is disposed within the support structure 102. In another embodiment, no greater than 100% of the first arm engagement portion 126 is disposed within the support structure 102.

In an embodiment, the first arm engagement portion 126 includes a generally uniform composition around the entire circumference of the first arm engagement portion 126. In another embodiment, the first arm engagement portion 126 can include a first material at a first location and a second material at a second location, the first and second locations and materials being different from one another.

In an embodiment, the first arm engagement portion 126 comprises an elastic material adapted to deform upon application of a force. In another embodiment, the first arm engagement portion 126 comprises an inelastic, or generally inelastic, material. In yet a further embodiment, the first arm engagement portion 126 can include at least two different materials having different moduli of elasticity as compared to one another. In a particular embodiment, portions of the first arm engagement portion 126 disposed within the support structure 102 can include materials having a lower elastic modulus as compared to exposed portions of the first arm engagement portion 126. In another particular embodiment, portions of the first arm engagement portion 126 disposed within the support structure 102 can be inelastic, or generally inelastic. In a further particular embodiment, exposed portions of the first arm engagement portion 126 can be elastic. In an embodiment, at least 1% and no greater than 99% of the first arm engagement portion 126 comprises an elastic material, or at least 2% and no greater than 70% of the first arm engagement portion 126 comprises an elastic material, or at least 2% and no greater than 40% of the first arm engagement portion 126 comprises an elastic material, or at least 2% and no greater than 15% of the first arm engagement portion 126 comprises an elastic material.

In an embodiment, the first arm engagement portion 126 or first engagement feature 112 can include a slip reducing element (not illustrated) adapted to reduce movement of the first arm engagement portion 126 relative to the shoulder. In another embodiment, the first arm engagement portion 126 or first engagement feature 112 can include a slip enhancing element (not illustrated) adapted to promote movement of the first arm engagement portion 126 relative to the shoulder. In yet a further embodiment, the first arm engagement portion 126 or first engagement feature 112 can include a slip reducing element at a first location and a slip enhancing element at a second location. In an embodiment, at least one of the slip reducing or slip enhancing elements can be detachable or repositionable. In another embodiment, at least one of the slip reducing or slip enhancing elements can be changeable between a number, n, of uses (e.g., between successive uses where n equals 1 or after multiple uses where n is greater than 1), permitting introduction of a new slip reducing or slip enhancing element after the number, n, of uses.

In a non-illustrated embodiment, the first arm engagement portion can be formed, at least in part, by one or more portions of the support structure. For example, at least part of the first arm engagement portion 126 contained within the shaded region in FIG. 4 can be defined by a portion of the support structure 102. As illustrated in FIG. 4, the first arm engagement portion 126 is formed from a different element than the support structure 102 and is embedded within the support structure 102.

In an embodiment, the support structure 102 can include a number of discrete layers, n, as measured by a number of readily discernable layers, as viewed in cross section, at a location spaced apart from the first arm engagement portion 126. The garment 100 can include a number of layers, n+x, as measured at the first arm engagement portion 126, where x is a unit integer of at least 1. That is, in an embodiment, the first arm engagement portion 126 can increase material ply of the support structure 102 by at least one layer at locations shared by the first arm engagement portion 126 and support structure 102.

Referring to FIG. 2, the garment 100 can further include a second arm engagement portion 152 connected to the second engagement feature 114. The second arm engagement portion 152 can have any number of similar or different attributes as described herein with respect to the first arm engagement portion 126. In a particular embodiment, the first and second arm engagement portions 126 and 152 are reflectively symmetrical or capable of being reflectively symmetrical about a vertical axis—e.g., the spine, when similarly sized. In another embodiment, the first and second arm engagement portions 126 and 152 are not symmetrical with one another.

In an embodiment, the first end 120 of the first engagement feature 112 is at a lower vertical elevation than the second end 122 of the first engagement feature 112. In an embodiment, the first engagement feature 112, or a best fit line thereof, can form an angle, α, with respect to flat, level ground on which the wearer is standing when wearing the garment 100. In a particular embodiment, the angle, α, can be at least 1°, or at least 2°, or at least 5°, or at least 10°, or at least 25°, or at least 45°. In another embodiment, the angle, α, can be no greater than 90°, or no greater than 75°, or no greater than 60°. In a further embodiment, the angle, α, can be in a range of 1° and 89°, or in a range of 5° and 80°, or in a range of 10° and 60°, or in a range of 15° and 50°, or in a range of 20° and 45°. In an embodiment, the angle, α, is a positive (upward) angle.

In an embodiment, the first engagement feature 112 can lie along two or more lines, or best fit lines, that intersect one another. For example, in a particular, non-illustrated embodiment, a first portion of the first engagement feature 112 can extend from the support structure 102 along a first best fit line toward the spine. A second portion of the first engagement feature 112 can extend along a second best fit line oriented upward toward the shoulder at a different angle than the first line, with respect to the ground. The angle between the first and second portion can be in a range of 1° and 179°, or in a range of 60° and 175°, or in a range of 90° and 170°, or in a range of 95° and 160°, or in a range of 100° and 155°, or in a range of 105° and 145°. In an embodiment, the interface between the first and second portions of the first engagement feature 112 can be an arcuate interface. In another embodiment, the interface between the first and second portions can be an angled interface including two line segments meeting at a relative angle. In an embodiment, the first and second portions can be coupled directly together. In another embodiment, the first and second portions can be unitary. In another embodiment, the first and second portions can be connected together through an intermediary element. The intermediary element can include, for example, an elastic material or a rigid material having an attribute adapted to enhance the position of the first and second portions relative to one another, a ring, or any combination thereof. In an embodiment, the intermediary element is an adjustable element such that the relative position or angle of the first and second portions can be adjusted.

FIG. 4 illustrates an edge 154 of the support structure 102 extending between the band 136 and the first arm engagement portion 126. In the illustrated embodiment, the edge 154 extends backward and upward from the band 136 to the first arm engagement portion 126. In another embodiment, the edge 154 can extend forward and upward from the band 136 to the first arm engagement portion 126. In a further embodiment, the edge 154 can extend directly upward from the band 136 to the first arm engagement portion 126. In an embodiment, the edge 154 can include a linear segment. In another embodiment, the edge 154 can include an arcuate segment. In a further embodiment, the edge 154 can include a linear segment and an arcuate segment. Connection points between the edge 154 and the first arm engagement portion 126 and band 136 can be disposed further forward or rearward as compared to the illustrated figure.

An opening 156 in the garment 100 defined, for example, by portions of the edge 154, the band 136, first arm engagement portion 126, and second engagement feature 114 can permit greater garment flexure as compared to garments without similar openings. The opening 156 can have a shape when worn on the body that is generally arcuate, generally polygonal, or that includes generally arcuate and generally polygonal perimeter segments. By way of non-limiting example, the opening 156 can be generally ovular, triangular, quadrilateral, pentagonal, or tear drop shaped. In an embodiment, the garment can include one or more interconnects (not illustrated) extending at least partially between edges of the opening 156. By way of non-limiting example, at least one of the one or more interconnects can extend between the first arm engagement portion 126 and the second engagement feature 114 or between the second arm engagement portion 126 and the edge 154 of the support structure 102 or between the second engagement feature 114 and the edge 154. In a particular instance, the interconnects can alter force loading of the garment along the body, provide an attachment point for another element (such as another part of the garment or a discrete component attachable thereto), enhance garment aesthetic, provide force against the body of the wearer (e.g., inward compression), provide indicia or a location where indicia can be added to the garment, increase posture enhancing effects of the garment, or any combination thereof. In an embodiment, the interconnect can include a fabric, such as mesh or webbing, cord, non-mesh fabric, or any combination thereof.

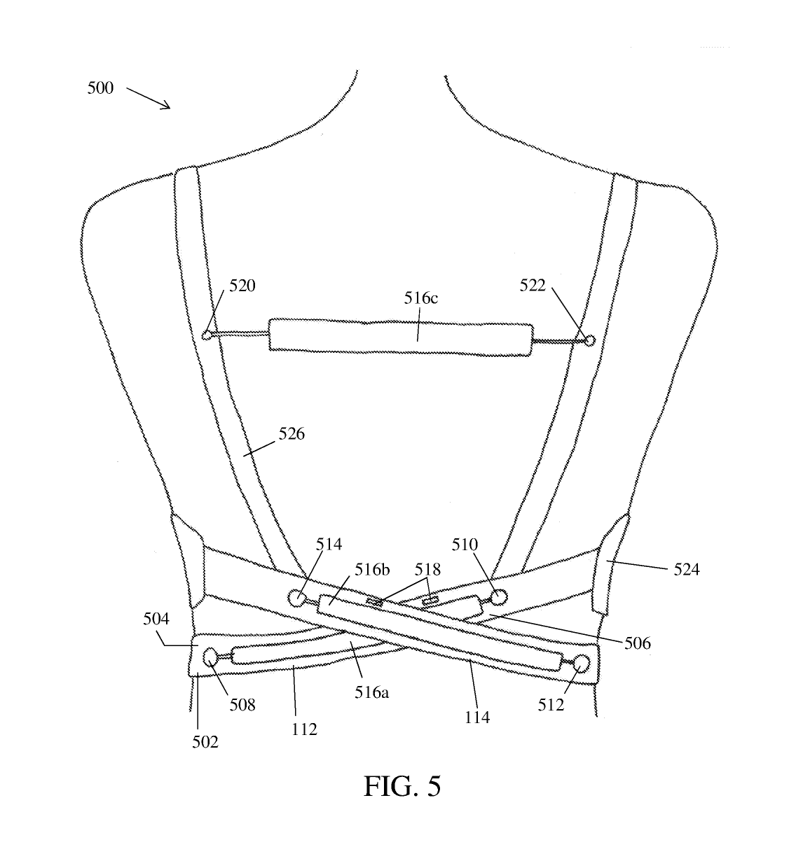

Referring to FIG. 5, in a particular embodiment, the first engagement feature 112 can include a plurality of zones, such as zones 502, 504, and 506, having different responses during application of force (e.g., different elasticity). This can enable loading and transmission of forces along predetermined paths of the first engagement feature 112 while reducing loading and transmission of forces along other paths of the first engagement feature 112. The zones can be disposed along the first engagement feature 112 at any relative position with respect to one another to cause a desired characteristic. In an embodiment, the zones are disposed along a line (e.g., zone 504 is disposed between zones 502 and 506). In another embodiment, the zones are not disposed along a line (e.g., zone 502 contacts zones 504 and 506, zone 504 contacts zones 502 and 506, and zone 506 contacts zones 502 and 504).

In an embodiment, the first engagement feature 112 can include coupling points, such as coupling points 508 and 510 adapted to couple a component 516a to the first engagement feature 112. The second engagement feature 114 can include coupling points 512 and 514 adapted to couple a component 516b to the second engagement feature 114. In an embodiment, at least one of coupling points 508, 510, 512, and 514 is disposed along the support structure 524 or arm engagement portion 526. In another embodiment, at least one of the components 516a, 516b, or 516c (described below) can attach to the garment 500 without a coupling point (e.g., using a clip, at least partially wrapping the component through an opening, etc.).

The garment 100 can further include one or more coupling points (e.g., coupling points 520 and 522) disposed at locations spaced apart from the first and second engagement features 112 and 114. In an embodiment, the coupling points 520 and 522 can be disposed on the arm engagement portions 526. As illustrated in FIG. 5, coupling points 520 and 522 can be coupled together by a component 516c. The coupling points 520 or 522 can also be coupled by a component to coupling points 508, 510, 512, or 514.

In an embodiment, at least two of the components 516a, 516b, and 516c can have similar attributes as compared to one another. For example, at least two of the components 516a, 516b, and 516c can include a same elasticity, a same size, a same weight, a same shape, a same color, the same of another attribute, or any combination thereof. In another embodiment, at least two of the components 516a, 516b, and 516c can have one or more different attributes as compared to one another. For example, components 516a and 516c can differ in elasticity, size, weight, shape, color, other attributes, or any combination thereof.

In an embodiment, at least one of the components 516a, 516b, or 516c can include a display (e.g., a banner) with optional indicia, a rigid or semi-rigid component, a loaded component having a pre-stress adapted to bias the garment 500, or any combination thereof. In a particular embodiment, at least one of the components 516a, 516b, or 516c can be interchangeable such that different components 516a, 516b, or 516c can be installed without requiring different attachment protocol. In this regard, a wearer can change between components to alter the feel or function of the garment. By way of a non-limiting example, at least one of the coupling points 508, 510, 512, 514, 520, and 522 can include a clasp, a hook, a button, a snap, a buckle, other readily known coupling protocol, or any combination thereof. When not engaged with a component 516a, 516b, or 516c, at least one of the coupling points 508, 510, 512, 514, 520, and 522 can be moveable, detachable, or hidden. In a particular embodiment, at least one of the components 516a, 516b, or 516c is, or can be, non-detachably coupled to the garment 500.

In an embodiment, one or more sensors 518 can be disposed on the garment 500. For example, one or more sensors 518 can be disposed on the first engagement feature 112. In a particular embodiment, the one or more sensors 518 can detect force applied on the shoulders by the first engagement feature 112. In another embodiment, the one or more sensors 518 can detect relative X-, Y-, or Z-orientations of the first engagement feature 112 to permit detection of bodily alignment (e.g., proper posture). In yet another embodiment, the one or more sensors 518 can detect force applied between any two or more locations of the garment 100 (e.g., between any two of coupling points 508, 510, 512, 514, 520, and 522). In a particular embodiment, at least one of the sensors 518 is a micro-electrical-mechanical (MEM) sensor. In an embodiment, the one or more sensors 518 can be in communication with a logic element to determine a desired attribute of the garment 100 or wearer from data obtained by the one or more sensors 518. In another embodiment, the one or more sensors 518 can be in communication with device, such as a speaker, a Bluetooth or Wi-Fi device, or any combination thereof. In a particular instance, the device(s) can record the data obtained by the sensor 518 or provide a user with real-time or post-time feedback.

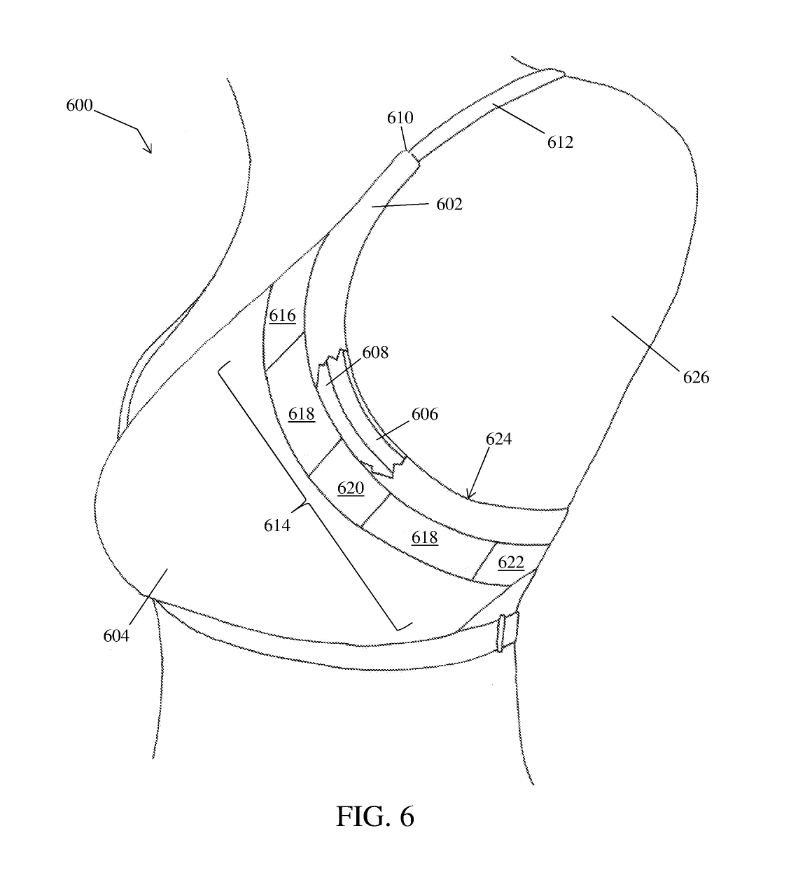

FIG. 6 illustrates a partially cut away view of a garment 600 in accordance with another embodiment. The garment 600 includes an arm engagement portion 602 and a support structure 604. The arm engagement portion 602 includes a component 606 disposed at least partially within an opening 608 of the support structure 602. In an embodiment, the component 606 is adapted to float within the opening 608. In another embodiment, the garment 600 includes one or more locking features (not illustrated) adapted to selectively prevent movement (e.g., translation or rotation) of the component 606 within the opening 608. In yet a further embodiment, the component 606 is statically fixed relative to the support structure 604 at one or more locations. In an embodiment, the component 606 can be disposed along an upper edge 624 of the support structure 602. In another embodiment, the component 606 can be spaced apart from the upper edge 624.

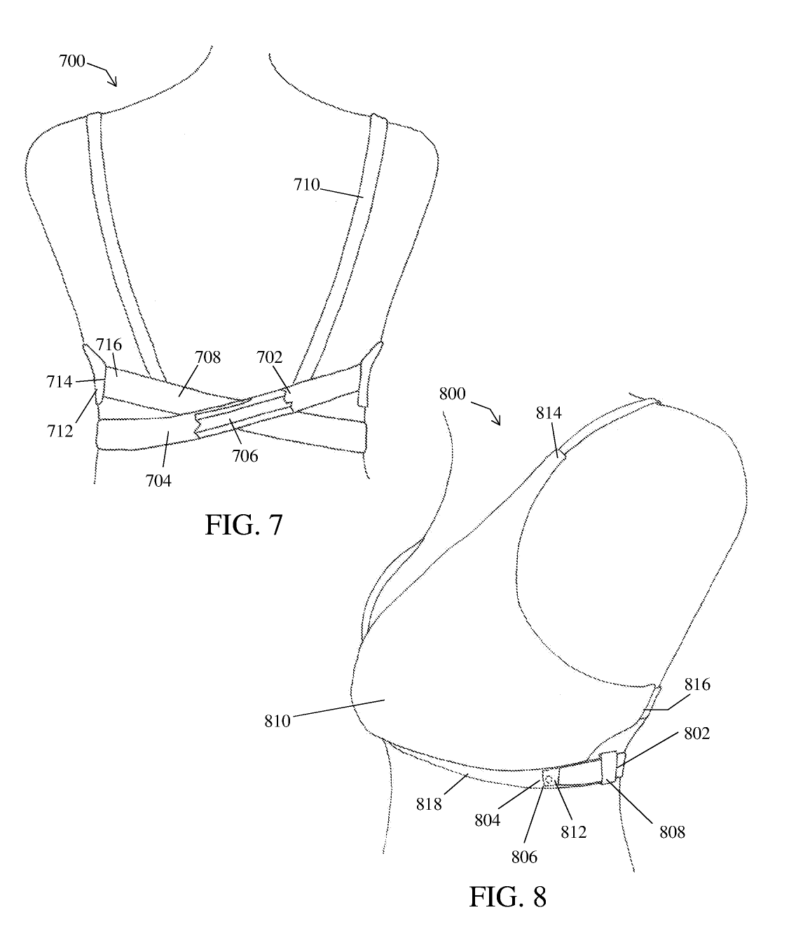

In an embodiment, the component 606 extends between a first location 610 and a second location 714 (FIG. 7) of the garment 600. In another embodiment, the component 606 can extend less than an entire distance between the first and second locations 610 and 714. The component 606 can be coupled to the sides of the opening 608. In a particular instance, the component 606 can extend around no greater than 90% of the circumference of opening 626, no greater than 50% of the circumference, no greater than 25% of the circumference, no greater than 10% of the circumference, no greater than 5% of the circumference, or no greater than 2% of the circumference.

The component 606 can be coupled with other portions 612 and 716 (FIG. 7) of the arm engagement portion 602 at or adjacent locations 610 and 714. In another embodiment, the component 606 and other portions 612 and 716 of the arm engagement portion 602 can have a unitary construction. In a particular instance, at least one of the component 606, other portion 612, and other portion 716 of the first arm engagement portion 602 can be coupled with the support structure 602 to mitigate relative movement therebetween which could cause skin irritation or garment misalignment. In another instance, the support structure 602 can be at least partially disconnected from the component 606, the other portion 612, the other portion 716, or any combination thereof to enhance structural or aesthetic functionality.

In an embodiment, the support structure 604 is adapted to remain relatively fixed with respect to the body while the component 606 moves and flexes, or vise versa. In such a manner, the garment 600 can flex and move without occurrence of skin irritation, such as chaffing.

In an embodiment, the garment 600 can include a connective portion 614. The connective portion 614 can include, for example, a textile, organic material, polymer, or any combination thereof. In a particular embodiment, the connective portion 614 is mesh. In a particular embodiment, the connective portion 614 includes a material having different material property than the first and second engagement features and the support structure 604. In an embodiment, the connective portion 614 can connect portions of the garment 600 together. This can, for example, prevent garment entanglement when the garment 600 is not in use, provide additional support, enhance aesthetic appeal of the garment, or any combination thereof.

In an embodiment, the connective portion 614 has a unitary construction. In another embodiment, the connective portion 614 includes a multiple-piece construction. In an embodiment, the connective portion 614 is multi-ply. The multi-ply connective portion 614 can include a first layer of a first material and a second layer of a second material. In a particular embodiment, the first and second materials are the same as one another. In another particular embodiment, the first and second materials are different than one another. In an embodiment, the first and second layers can operate with different loading conditions when the garment 600 is in use. For example, the first layer may be adapted to handle higher loading forces than the second layer. In such a manner, the first layer may provide a biasing force to the garment while the second layer provides a different property to the garment 600. The first and second layers can be attached together at one or more locations along the connective portion 614. In an embodiment, at least a portion of the first and second layers float relative to one another. In such a manner, the first and second layers can move, stretch, or otherwise deform independently at the floating locations. In an embodiment, the connective portion 614 includes a plurality of zones (e.g., zones 616, 618, 620, and 622). In a particular embodiment, at least two of the zones can have different properties as compared to one another. For example, the zones can have different elasticity, different size, different ply, different shape, different material composition, different moisture wicking ability, or any combination thereof. By way of a non-limiting example, zones 618 and 618 can have a first material property and zones 616, 620, and 622 can have a second material property different from the first material property.

While zones 616, 618, 620, and 622 are illustrated in FIG. 6 in a row near a lateral-upper edge of the support structure 604, it is noted that the zones can also be positioned at other locations along the support structure 604, the engagement features, the arm engagement portions, or any combination thereof. Further, while two or more zones may contact one another, the zones can also be spaced apart from one another in non-linear arrangement to achieve a particular areal effect (e.g., moisture wicking capability, strength, aesthetic, etc.).

In an embodiment, the zones of the connective portion 614 can be spaced apart from one another by portions of the support structure 604, portions of the arm engagement portion 624, or other components of the garment 600.

FIG. 7 illustrates a rear view of a garment 700 in accordance with an embodiment. The garment can include a first engagement feature 702. As illustrated, the first engagement feature 702 can have a multi-piece construction. For example, the first engagement feature 702 can include an outer component 704 and an inner component 706 disposed at least partially, such as fully, within the outer component 704.

The outer component 704 can cover at least a portion, such as all, of the inner component 706. In an embodiment, the outer component 704 can cover at least 1% of the inner component 706, or at least 10% of the inner component 706, or at least 25% of the inner component 706, or at least 50% of the inner component 706, or at least 90% of the inner component 706. In another embodiment, the outer component 704 can cover less than 99% of the inner component 706.

In an embodiment, the garment 700 is adapted to transmit force to the arm engagement portion 710 from the support structure 712 primarily through the inner component 706 of the first engagement feature 702. While the outer component 704 may transmit force, the garment 700 can be adapted such that at least 10% of transmitted force is transmitted through the inner component 706, or at least 51% of transmitted force is transmitted through the inner component 706, or at least 75% of transmitted force is transmitted through the inner component 706, or at least 99% of transmitted force is transmitted through the inner component 706.

In a particular embodiment, the inner component 706 of garment 700 (FIG. 7) can be coupled with the component 606 of garment 600 (FIG. 6). In a more particular embodiment, the inner component 706 can be unitary with the component 606. In another embodiment, the inner component 706 is not directly coupled with the component 606.

In an embodiment, the inner component 706 can emerge from the outer component 704. In another embodiment, a wearer can adjust tension or position of the inner component 706 without altering tension of the outer component 704. By way of example, the inner component 706 can emerge from the outer component 704 at a location adjacent to the side or front of the body. The inner component 706 can be secured to the support structure 712 via static or dynamic engagement to permit fixed or selective coupling, respectively, of the inner component 706 by a user.

In a particular instance, the outer component 704 can include a water-resistant or waterproof material or coating adapted to reduce penetration of sweat into the inner component 706. In another embodiment, the outer component 706 can include one or more fluid wicking perforations. In an embodiment, the outer and inner components 704 and 706 can be visually differentiated, such as for example, by color, size, shape, texture, or any combination thereof.

FIG. 8 illustrates a side view of a garment 800 in accordance with an embodiment including a first engagement feature 802. In a previously described embodiment, the first engagement feature is statically coupled to the support structure (FIG. 1). As illustrated in FIG. 8, the first engagement feature 802 can be dynamically coupled to the support structure 810 or band. In such a manner, the length, position, orientation, or any other similar adjustable attribute of the first engagement feature 802 with respect to the support structure 810 can be adjusted. In a particular embodiment, the first engagement feature 802 is coupled to the support structure 810 by an adjustable element 808. The adjustable element 808 can allow for adjustment of the length of the first engagement feature 802 or permit a user to selectively tighten or loosen the garment 800 before or after wearing. In a particular embodiment, the adjustable element 808 can include a one-way engagement feature adapted to permit one-way motion while selectively preventing motion of the first engagement feature 802 (or any portion associated therewith) in the other direction. For example, in an embodiment, the one-way engagement element can permit forward-oriented motion (or rearward-oriented motion) of the first engagement feature 802 while resisting, or preventing, undesired rearward (or forward) motion thereof. Thus, a user can pull the first engagement feature 802 through the one-way engagement element in a forward direction to tighten the garment 800 without the first engagement feature 802 undesirably retracting and loosening. In a particular embodiment, the adjustable element 808 can be releasable such that a user can selectively release the first engagement feature 802 from the support structure 810.

In an embodiment, the garment 800 includes a receiving portion 812 adapted to receive a portion (such as an end 804) of the first engagement feature 802, for example, when the garment 800 is in use. In certain instances, the end 804 of the first engagement feature 802 can extend past the adjustable element 808. Thus, the receiving portion 812 can accommodate the end 804 of the first engagement feature 802 to prevent undesirable movement thereof. In a particular embodiment, the receiving portion 812 includes a pocket, a strap, a loop, a hook and loop engagement interface, a clasp, complementary elements, or any combination thereof. In an embodiment, the receiving element 812 is disposed below, or at the bottom of, the support structure 810. In another embodiment, the receiving element 812 is disposed on one of the cups of the support structure 810, such as within, in front of, or behind the first cup.

In an embodiment, the garment 800 can include one or more rerouting elements (not illustrated) such as rings (e.g., O-rings, D-rings, or the like) or loops through which the first engagement feature 802 can extend. The one or more rerouting elements can change the angle the user adjusts the first engagement feature 802 relative to the support structure 810. For example, as described above, a user can pull the end 804 of the first engagement feature 802 forward to tighten the first engagement feature 802. In embodiments with one or more rerouting elements, the direction of adjustment can be modified. Thus, for example, a user can tighten the first engagement feature 802 by pulling the end 804 in a direction other than forward, such as downward, upward, or rearward. In a particular embodiment, at least one of the one or more rerouting elements can be at least partially hidden such that a viewer cannot see at least a portion of the at least one of the one or more rerouting elements. In another embodiment, at least one of the one or more rerouting elements can be detachable or adjustable with respect to its/their location on the garment 800. In yet a further embodiment, at least one of the one or more rerouting elements can be interchangeable such that a shape, size, or type of rerouting element can be modified.

In an embodiment, the end 804 of the first engagement feature 802 can include a detent 806 adapted to prevent complete disengagement of the end 804 of the first engagement feature 802 from the support structure 810. The detent 806 can be shaped or sized such that the end 804 of the first engagement feature 802 is not removable past a complementary portion of the garment 800. For example, the adjustable element 808 can include an opening through which the first engagement feature 802 extends and through which the detent 806 cannot pass. In an embodiment, the detent 806 can be removable such that a user can selective pass the end 804 of the first engagement feature 802 through the opening, such as, for example, when cleaning or modifying the configuration of the garment 800. It is noted that the adjustable element 808, one or more rerouting elements, the receiving portion, or any combination thereof can be positioned at other locations along the garment 800, such as at locations 814 or 816.

In a non-illustrated embodiment, adjustment of the force applied by the first engagement feature 802 to the opposite shoulder of the wearer can be adjusted by adjusting the band 818. In another non-illustrated embodiment, adjustment of the force applied by the first engagement feature 802 to the opposite shoulder of the wearer can be adjusted by an element disposed along the front of the garment 100. Adjusting the element can pull the end 804 (or a location proximate thereto) forward relative to the body, thus adjusting the force applied by the first engagement feature 802 on the opposite shoulder.

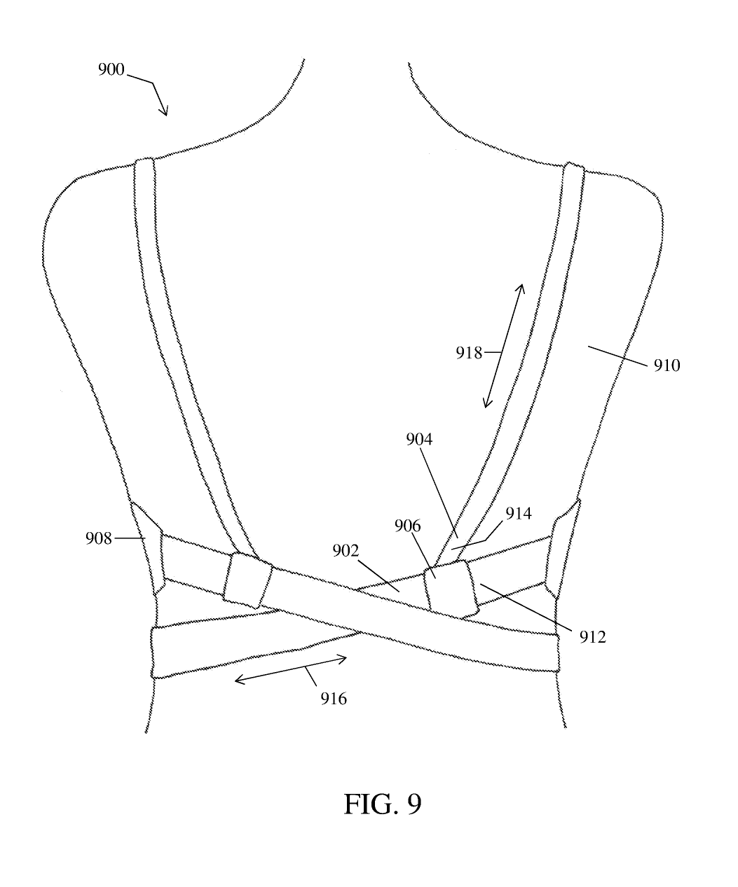

FIG. 9 illustrates a rear view of a garment 900 in accordance with another embodiment including a first engagement feature 902 and a first arm engagement portion 904. An interface 906, such as a slide, buckle, clip, snap, or any combination thereof, can be disposed between, or engage, the first engagement feature 902 with the first arm engagement portion 904.

In an embodiment, the interface 906 is repositionable so as to permit reshaping or resizing of an opening 910 defined by the first arm engagement portion 904. For example, in an embodiment, the interface 906 can be moved along one or both of the first engagement feature 902 and the first arm engagement portion 904, thereby adjusting the shape or size of the opening 910. In a particular instance, the interface 906 is adapted to slide in a direction generally parallel with line 916 which is generally parallel with the first engagement feature 902. In another instance, the interface 906 is adapted to slide in a direction generally parallel with line 918 which is generally parallel with respect to an adjacent portion of the first arm engagement portion 904. In yet a further instance, the interface 906 can slide in a direction formed in part by both lines 916 and 918.

In an embodiment, the interface 906 is detachable or attachable with respect to one or both of the first engagement feature 902 and first arm engagement portion 904, permitting adjustment of size or shape of the opening 910.

In an embodiment, the first arm engagement portion 904 includes a single element extending from location 912 to location 914 around the shoulder of the wearer. The first arm engagement portion 904 can be coupled with or unitary with the first arm engagement portion 904 at or adjacent to location 912. In such a manner, the illustrated first engagement feature 902 and first arm engagement portion 904 extend counter-clockwise from the support structure 908 around the opposite lateral side shoulder. In an embodiment, the orientation of the first arm engagement portion 904 can be switched from counter-clockwise to clockwise, for example, by releasing the interface 906 and rerouting the first engagement feature 902 and first arm engagement portion 904. Orientation of the first engagement feature 902 and first arm engagement portion 904 relative to the shoulder can determine the angle of force applied on the shoulder. Some wearers may prefer a clockwise orientation while other wearers may prefer a counterclockwise orientation. Garments in accordance with embodiments described herein can include clockwise orientation, counter-clockwise orientation, or reversibility between clockwise and counter-clockwise orientations.

FIG. 10 illustrates an embodiment of a garment 1000 including a first engagement feature 1002 and a second engagement feature 1004. In a first configuration (illustrated by solid lines) the first engagement feature 1002a extends from a first lateral side 1006 of a support structure 1008 of the garment 1000 to the shoulder of the second lateral side 1008 of the body. In the first configuration the second engagement feature 1004a extends from a second lateral side 1010 of the support structure 1008 to the shoulder of the first lateral side of 1006 of the body.

The first and second engagement features 1002a and 1004a can each include one or more disconnectable elements 1012. The first and second engagement features 1002a and 1004a can be selectively adjusted to a second configuration by disconnected the disconnectable elements 1012, repositioning the first and second engagement features 1002a and 1004a and reconnecting the disconnectable elements 1012. In the second configuration (illustrated by dashed lines) the first and second engagement features 1002b and 1004b do not cross over one another. Instead, the first and second engagement features 1002b and 1004b extend horizontally, or generally horizontally. In a particular embodiment, the first and second engagement features 1002b and 1004b are parallel, or generally parallel, with respect to one another when in the second configuration. Adjusting between the first and second configurations may modify the force vectors of the engagement features 1002 and 1004 on the shoulders of the body. For example, in the first configuration the garment 1000 can provide greater downward, rearward, or inward force to the shoulders as compared to the garment in the second configuration. Thus, the first configuration may improve posture and reduce shoulder fatigue while the second configuration may provide reduced forces to the shoulders.

In an embodiment, the disconnectable elements 1012 are readily disconnectable such that a user can rapidly adjust between the first and second configurations described above. In an embodiment, the garment 1000 is adjustable between the first and second configurations while on the body of the wearer. For example the disconnectable elements 1012 can be connectible, or reconnectable, while the garment 1012 is on the body of the wearer. In an embodiment, the disconnectable elements are disconnectable upon application of a force in a range of 0.1 N and 100 N, or in a range of 1 N and 50 N, or in a range of 1 N and 10 N. In another embodiment, the disconnectable elements are disconnectable upon application of a hand generated force.

In an embodiment, at least one of the disconnectable elements 1012 includes a first part 1016 and a second part 1018. In a particular embodiment, the first parts 1016 of the disconnectable elements 1012 can be identical, or nearly identical, with respect to one another. In another particular embodiment, the second parts 1018 of the disconnectable elements 1012 can be identical, or nearly identical, with respect to one another. A wearer can adjust the disconnectable elements 1012 to configure the garment 1000 in either the first or second configurations.

In a particular instance, the disconnectable element 1012 is dynamically coupled to the first engagement feature 1002 such that the disconnectable element 1012 is adjustable in position with respect to the first engagement feature 1002. This may permit adjustment of the length of the first engagement feature 1002 without extra hardware. In another instance, the disconnectable element 1012 is fixedly coupled to the first engagement feature 1002, such as at an end of a first part 1022 of the first engagement feature 1002. In this regard, the disconnectable element 1012 can be permanently positioned with respect to the end of the first part 1022. Dynamic or fixed coupling can occur at either end of either the first and second engagement features 1002 or 1004.

In an embodiment, the garment 1000 can be fixedly positioned in the first configuration (i.e., not adjustable to the previously described second configuration). In another embodiment, the garment 1000 can be fixedly positioned in the second configuration (i.e., not adjustable to the previously described first configuration). In the latter embodiment, the formed upper arm engagement portion can be adapted to provide posture enhancing force to the shoulder, for example using an adjustable element such as a slide to permit tightening and loosening of the garment. Independent adjustment of the upper and lower arm engagement portions can permit posture enhancing benefits while simultaneously keeping the garment secure on the body.

In an embodiment, the garment 1000 is adapted to include a third configuration (not illustrated). In the third configuration, at least one of the first and second engagement features 1002 and 1004 extends between the lateral side 1006 or 1010 of the support structure 1008 and the arm engagement feature 1020 on the same lateral side 1006 or 1010 of the garment 1000. In a particular embodiment, the first or second engagement feature 1002 or 1004 extending between the support structure 1008 and the arm engagement feature 1020 on the same lateral side 1006 or 1010 of the body can include a disconnectable element (not illustrated), instead of or in addition to the disconnectable elements 1012 previously described, on the same lateral side 1006 or 1010 of the garment 1000.

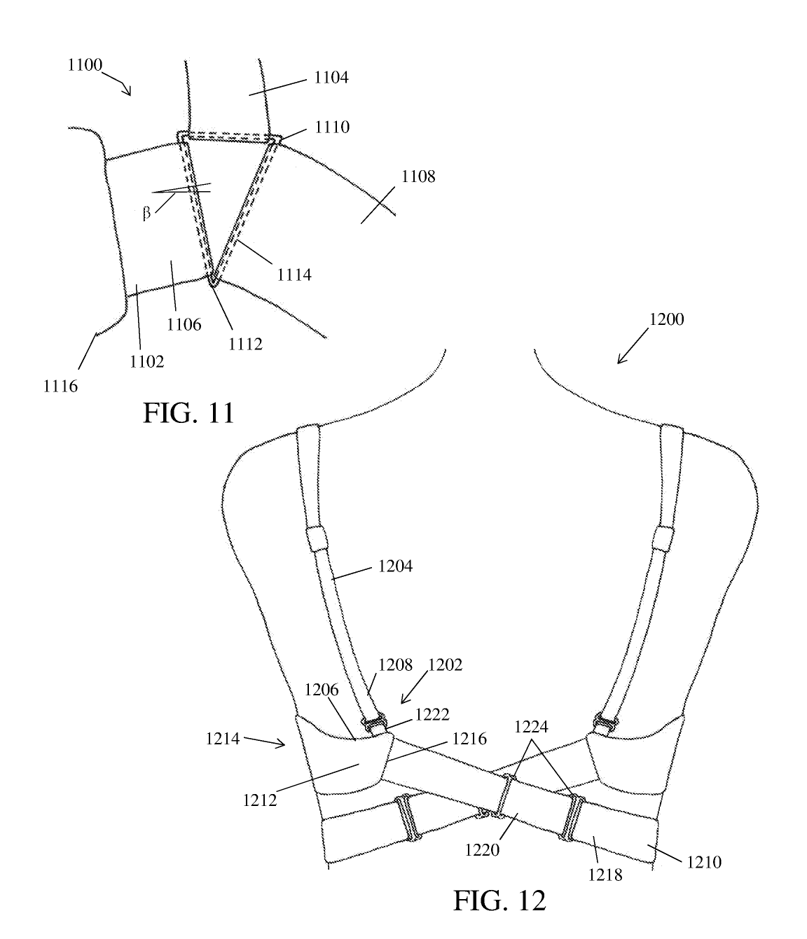

FIG. 11 illustrates an embodiment of a junction 1100 formed between an arm engagement portion 1102, at a first end 1104 and a second end 1106, and an engagement feature 1108. In a further embodiment, the junction 1100 can include the support structure 1116. In the illustrated embodiment, the junction 1100 is maintained by a loop 1110. In a particular embodiment, the loop 1110 can have a triangular shape. In an embodiment, the loop 1110 is non-equilateral (isosceles or scalene). In another embodiment, the loop 1110 is equilateral. In other embodiments, the loop 1110 can have a different polygonal shape—such as rectangular or pentagonal. In yet other embodiments, the loop 1110 can have an arcuate shape—such as a generally circular shape. In an embodiment, use of the loop 1110 can eliminate additional hardware which may be required, for example, to use the adjustment element 132 (FIG. 2) or engagement feature 1108. In certain instances, garments including loops 1110 may exhibit reduced fabric distortion and warping, or conform better to the body than garments without loops.

In an embodiment, the loop 1110 is a textile material. In another embodiment, the loop 1110 is a polymer or metal. In an embodiment, exposed portions 1112 of the loop 1110 can have different attributes as compared to hidden portions 1114 of the loop 1110. For example, the exposed portions 1112 can be marked (e.g., color coded, patterned, or including symbols). By way of another example, the hidden portions 1114 can include a low friction material adapted to permit easier sliding of materials relative thereto.

In an embodiment, the loop 1110 can provide an interface to selectively move between first and second configurations of the garment (e.g., as discussed with respect to FIG. 10). For example, in an embodiment, the engagement feature 11108 can terminate at an element (not illustrated) adapted to selectively engage the loop 1110. The element 1110 can be detached from the loop 1110 and engaged to a corresponding component on the other engagement feature (not illustrated).

In a particular embodiment, the loop 1110 can be moveable, such as rotatable, between two or more different orientations. In a first orientation, the garment can have a first characteristic, such as a first shoulder-biasing force. In a second orientation, the garment can have a second characteristic different than the first characteristic.

In an embodiment, the junction 1100 can include a further component such as a button, snap, or the like, optionally with indicia; a band (detachable or fixed) extending to and selectively coupleable to, a different part of the garment or part of the wearer's body; or any other item which may functionally or aesthetically alter garment wearability. The second end 1106 of the arm engagement portion 1102 is illustrated in an upwardly angled orientation at the junction 1100. In an embodiment, the second end 1106 of the arm engagement portion 1102 can have an angle, β, as measured with respect to flat, level ground on which the wearer is standing when wearing the garment, that is at least 1°, or at least 5°, or at least 10°, or at least 15°, or at least 20°, or at least 45°. In an embodiment, angle β, is no greater than 150°, or no greater than 120°, or no greater than 90°. In an embodiment, angle β is less than 0°. That is, the angle can be downward.

In an embodiment, the junction 1100 can be devoid of a loop 1110. In a particular embodiment, the junction between the arm engagement portion and the engagement feature can have generally similar angles to those illustrated.

FIG. 12 illustrates an embodiment of a garment 1200 including a junction 1202 formed between an arm engagement portion 1204, at a first end 1206 and a second end 1208, and an engagement feature 1210. As illustrated, a portion 1212 of the support structure 1214 extends toward, and can extend to, the junction 1202. In an embodiment, the end 1208 of the arm engagement portion 1204 can be coupled with, such as embedded within, the portion 1212 of the support structure 1214. In another embodiment, the end 1208 of the arm engagement portion 1204 can be attached to a side or top of the portion 1212. In an embodiment, part of the arm engagement portion 1204 can include a different material than the remaining arm engagement portion 1204. For example, the arm engagement portion 1204 can include a portion 1222, for example disposed along end 1208, comprising a different material than the remaining arm engagement portion 1204. By way of non-limiting example, the portion 1222 can include a more elastic material as compared to the remaining arm engagement portion 1204. In a particular embodiment, the portion 1222 can be elastic while other exposed portions of the arm engagement portion 1204 are relatively inelastic. This can allow the arm engagement portion 1204 to stretch during bodily movement.

In an embodiment, the portion 1212, or part thereof, can be devoid of a discrete arm engagement portion coupled thereto, such as embedded therein. That is, in an embodiment, the portion 1212, or part thereof, can be devoid of a discrete arm engagement portion, for example in favor of the arm engagement portion being formed entirely by the support structure 1214 at the portion 1212, or part thereof.

In certain embodiments, the end 1208 of the arm engagement portion 1204 can be dynamically coupled with the junction 1202. For example, in a particular embodiment, the end 1208 can be pivotable with respect to the junction 1202. That is, the arm engagement portion 1204 can pivot clockwise and counter-clockwise with respect to the junction 1202 without binding or warping. In another embodiment, the end 1208 can be statically coupled with the junction 1202, mitigating pivoting which might otherwise occur. The end 1208 can also be removably, or otherwise dynamically, coupled with the junction 1202.

In an embodiment, an edge 1216 of the portion 1212 can be angled perpendicular to a best-fit centerline of the portion 1212. In another embodiment, the edge 1216 can be angled in a non-perpendicular orientation relative to a best-fit centerline of the portion 1212. In an embodiment, the edge 1216 can have a relative angle substantially equal to the angle of the engagement feature 1210. By way of non-limiting example, in an embodiment, the edge 1216 has an angle, as measured with respect to a vertical line, of at least 1°, or at least 5°, or at least 10°, or at least 20°, or at least 30°. In another embodiment, the edge 1216 has an angle no greater than 80°, or no greater than 60°, or no greater than 45°, or no greater than 35° with respect to the vertical line. As noted above, the engagement feature 1210 can have an angle, as measured with respect to a horizontal line, that is equal to the angle of the edge 1216. Thus, in an embodiment, the engagement feature 1210 can have an angle, with respect to a horizontal line, in a range between and including 1° and 80°. In an embodiment, a difference between the angle of the edge 1216 relative to a vertical line and the angle of the engagement feature 1210 relative to a horizontal line can be ±10°, or ±5°, or ±4°, or ±3°, or ±2°, or ±1°. The above-described angles of the engagement feature 1210 and edge 1216 can represent average anticipated angles as measured while the garment 1200 is on a wearer, mannequin, or the like.

In an embodiment, the end 1206 of the arm engagement portion 1204 can be embedded within the portion 1212 and extend at least partially, such as entirely, to the junction 1202. In a particular instance, the arm engagement portion 1204 can be joined to itself at the junction 1202 such that ends 1206 and 1208 are directly coupled together at the junction 1202. In an embodiment, the engagement feature 1210 can be coupled with, such as embedded within, the portion 1212 near the edge 1216.

In an embodiment, material (not illustrated), such as for example, mesh fabric, can extend between the engagement feature and the portion 1212. The material can extend from a location adjacent to a front or side of the garment 1200 and terminate prior to, at, or past the edge 1216.

In the illustrated embodiment, the engagement feature 1210 includes a first portion 1218 and a second portion 1220. The first and second portions 1218 and 1220 can have different material properties, such as different elasticities, different weights, different widths, different thicknesses, different material compositions, different aesthetics, or any combination thereof. In an embodiment, the first portion 1218 is more elastic than the second portion 1220. In a more particular embodiment, the second portion 1220 is relatively inelastic—having a stretch capacity no greater than 40%, or no greater than 30%, or no greater than 20%, or no greater than 10%. The engagement feature 1210 can include adjustable elements 1224 along the first portion 1218, the second portion 1220, or a combination thereof. At least one of the adjustable elements 1224 can include a grasping element (not illustrated) adapted to permit enhanced grip with the adjustable element 1224.

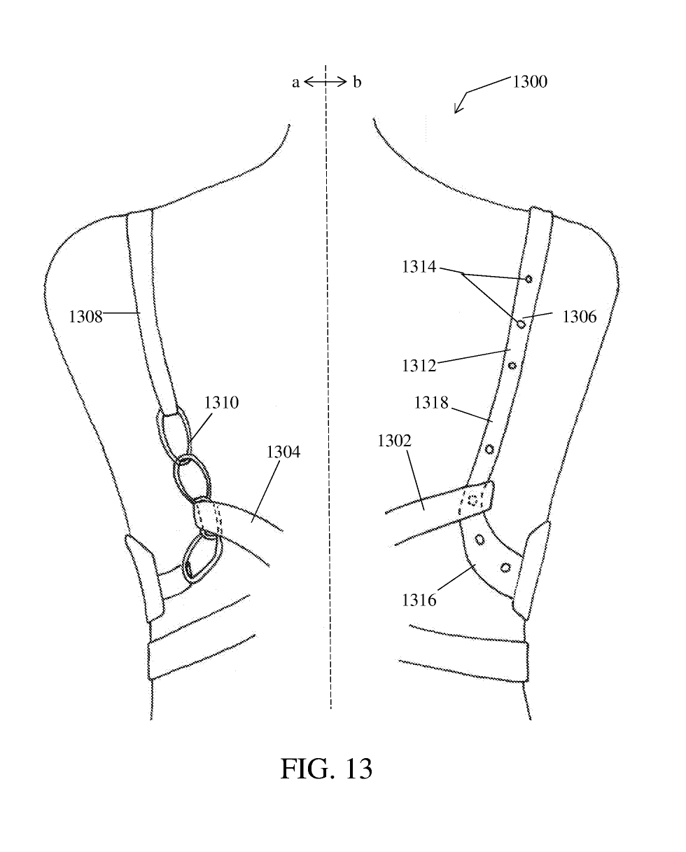

In an embodiment, the first and second engagement features are adjustable with respect to the first and second arm engagement portions. For example, an interface formed between the engagement feature and arm engagement portion can be adjustable. FIG. 13 includes two embodiments, a and b, of a garment 1300 where first and second engagement features 1302 and 1304 are adjustable with respect to the first and second arm engagement portions 1306 and 1308. In the first illustrated embodiment, marked as “a,” the second engagement feature 1304 engages with an adjustable portion 1310 of the second arm engagement portion 1308. The adjustable portion 1310 is illustrated as having a linked configuration. The second engagement feature 1304 can selectively couple with the adjustable portion 1310 at two or more locations along the adjustable portion 1310, such as at two or more of the illustrated links. In an embodiment, the adjustable portion 1310 includes at least two discrete adjustment locations, or at least three discrete adjustment locations, or at least five discrete adjustment locations, or at least ten discrete adjustment locations, or at least twenty discrete adjustment locations. In another embodiment, the adjustable portion 1310 includes no greater than 5000 discrete adjustment locations, or no greater than 1000 discrete adjustment locations, or no greater than 100 discrete adjustment locations, or no greater than 50 discrete adjustment locations. In another embodiment, the adjustable portion 1310 can have infinite adjustment capacity. For example, the adjustable portion 1310 can include no discrete engagement portions. Instead, the second engagement feature 1304 can be positioned at any relative location along the adjustable portion 1310. In a particular embodiment, the adjustable portion 1310 can be discernable from the rest of the second arm engagement portion 1308. In another particular embodiment, the adjustable portion 1310 can be part of the second arm engagement portion 1308 such that the adjustable portion 1310 is not visually discernable and identifiable.

The second engagement feature 1304 can attach to the adjustable portion 1310 via: a knot, a link or hook adapted to engage the adjustable portion 1310, a snap or button, a slide adapted to slide along the adjustable portion 1310, another known method, or using any combination thereof.

In the second illustrated embodiment, marked as “b,” the first engagement feature 1302 engages with an adjustable portion 1312 on the first arm engagement portion 1306 including a plurality of features 1314. By way of non-limiting examples, the features 1314 can include claps, snaps, buttons, knots, hook and loop engagement, openings, another known selective engagement system, or any combination thereof. In another embodiment, the adjustable portion 1312 can include loops or other features similar to the adjustable portion 1310.

In an embodiment, at least two pairs of the features 1314 can be spaced apart from one another by equal distances. In another embodiment, at least two pairs of features 1314 can be spaced apart from one another by unequal distances.

In an embodiment, the back of the garment can be symmetrical such that the interface formed by the engagement features and arm engagement portions is the same on both lateral sides of the body. In another embodiment, the back of the garment can be assymetrical such that the type of interface formed on the first lateral side is different than the type of interface formed on the second lateral side.

Force transmitted through the garment 1300 can vary at least in part based on the location of the interface between the engagement feature and arm engagement portion. In an embodiment, the adjustable portions 1310 or 1312 can allow a wearer to adjust the engagement features to a suitable angle for the wearer's body type. In a more particular embodiment, a wearer can adjust the relative tension/force applied in an underarm portion 1316 and overarm portion 1318 of the arm engagement portion. For example, adjusting the position of the interface between the engagement feature and arm engagement portion can change underarm:overarm force in a range of 100:0 and 0:100, or 90:10 and 10:90, or 80:20 and 20:80, or 70:30 and 30:70, or 60:40 and 40:60. In an embodiment, at least one possible interface location can cause a 50:50 split of force between the underarm and overarm portions 1316 and 1318.

In an embodiment, the underarm portion 1316 can include an adjustable interface (not illustrated), such as a releasable, slidable, or otherwise repositional interface, adapted to permit release or reduction of tension along the underarm portion 1316. In another embodiment, the overarm portion 1318 can include an adjustable interface (not illustrated), such as a releasable, slidable, or otherwise repositional interface, adapted to permit release or reduction of tension along the overarm portion 1318. The adjustable interfaces can be disposed on exposed portions of the arm engagement portion, within or on the support structure, or both.

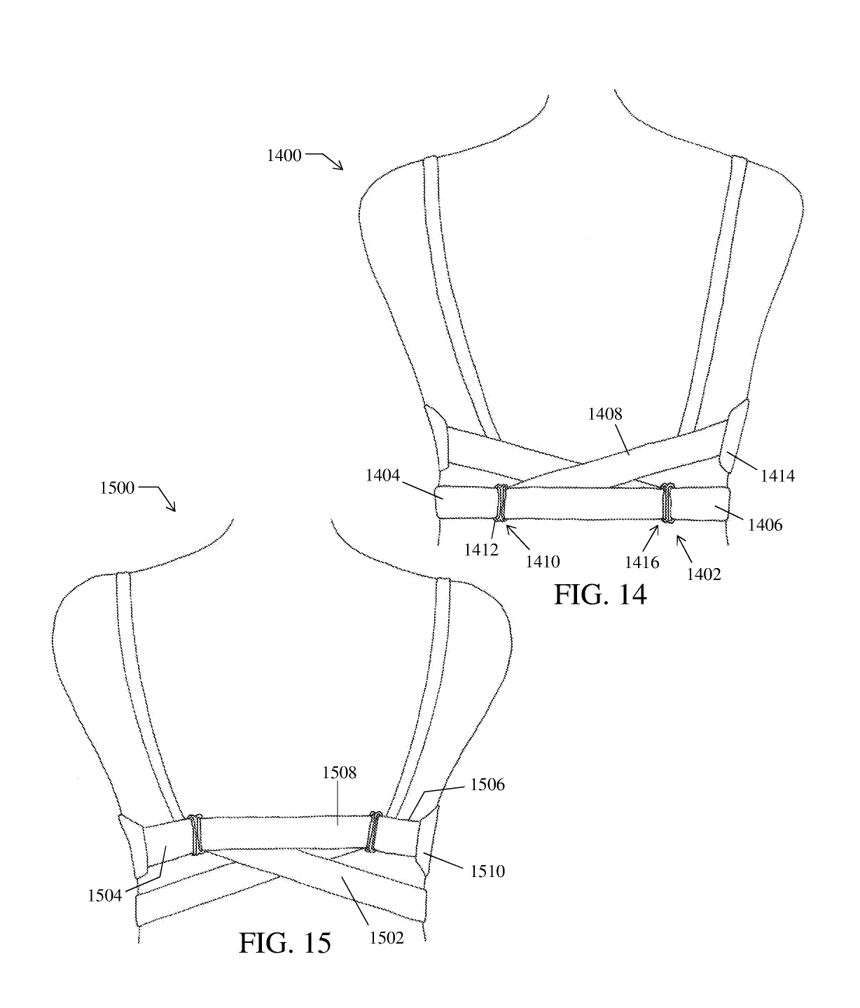

FIG. 14 illustrates a garment 1400 in accordance with an embodiment. The garment 1400 includes an engagement feature 1402 including a first portion 1404, a second portion 1406, and a third portion 1408. In the illustrated embodiment, the first and second portions 1404 and 1406 extend around lateral sides of the wearer. The third portion 1408 extends between the first and second portions 1404 and 1406. In an embodiment, the first, second, and third portions 1404, 1406, and 1408 can be unitary. In another embodiment, the first, second, and third portions 1404, 1406, and 1408 can include one or more components coupled together.

In an embodiment, engagement between the first portion 1404 and the third portion 1408 is dynamic For example, an interface 1410 between the first and third portions 1404 and 1408 can include a dynamic element 1412, adapted to permit engagement, such as dynamic engagement, of the first and third portions 1404 and 1408 relative to one another. The third portion 1408 can extend from the first portion 1404 to an opposite lateral side of the garment 1400, attaching, for example, at the support structure 1414 at the opposite lateral shoulder. Similarly, the second and third portions 1406 and 1408 can be dynamically engaged with one another at an interface 1416. The third portion 1408 can extend from the second portion 1406 to an opposite lateral side of the garment 1400, attaching for example, at the support structure 1414 at the opposite lateral shoulder.

At least one of the first, second, and third portions 1404, 1406, and 1408 can include an adjustable element adapted to adjust a length of the first, second, or third portion 1404, 1406, or 1408, respectively. In an embodiment, at least one of the first, second, and third portions 1404, 1406, and 1408 can include a disconnectable element (not illustrated) adapted to open and close the garment 1400 or permit adjustment between two or more configurations, as previously described.

FIG. 15 includes an illustration of a garment 1500 in accordance with an embodiment. The garment 1500 can be similar to garment 1400. Specifically, the garment 1500 can include an engagement feature 1502 including a first portion 1504, a second portion 1506, and a third portion 1508. Unlike the garment 1400 of FIG. 14, the first and second portions 1504 and 1506 can extend from the support structure 1510. The third portion 1508 can extend between the first and second portions 1504 and 1506 and engage with the support structure 1510 at a lower vertical elevation than the embodiment illustrated in FIG. 14. At least one of the first, second, and third portions 1504, 1506, and 1508 can include an adjustable element adapted to adjust a length of the first, second, and third portions 1504, 1506, and 1508, respectively. In an embodiment, at least one of the first, second, and third portions 1504, 1506, and 1508 can include a disconnectable element (not illustrated) adapted to open and close the garment 1500 or permit adjustment between two or more configurations, as previously described.

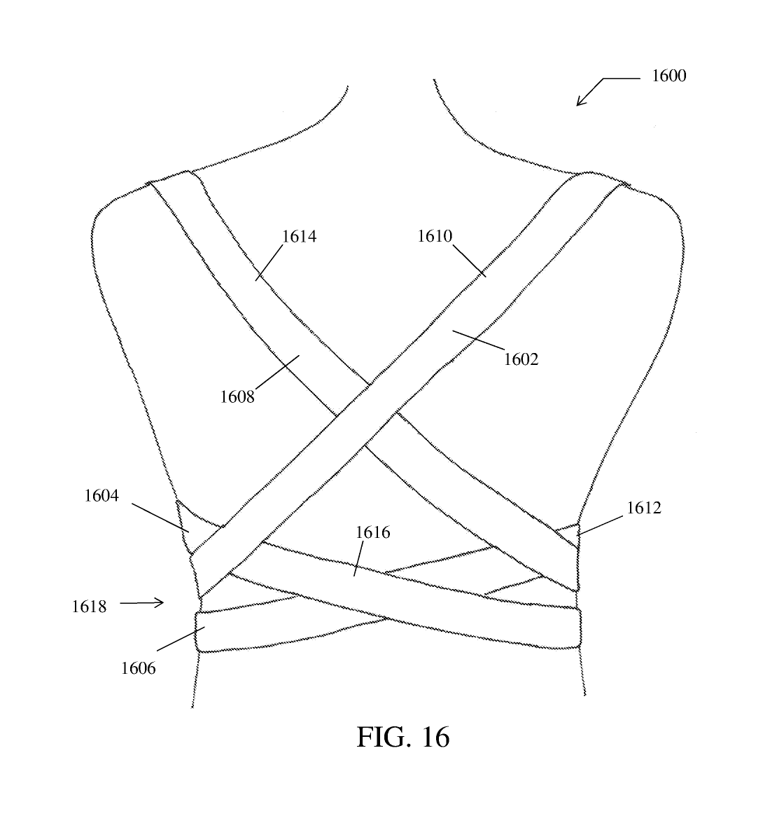

FIG. 16 illustrates a garment 1600 in accordance with an embodiment including a first engagement feature 1602 extending from a support structure 1618. The first engagement feature 1602 can extend from the support structure 1618, around the shoulder on the opposite lateral side of the body, and back to the support structure 1616. In an embodiment, the first engagement feature 1602 can include a length, L_{FEF}, as measured between opposite ends 1604 and 1606. In an embodiment, ends 1604 and 1606 can both terminate at a same lateral side of the support structure 1618. In a more particular embodiment, the ends 1604 and 1606 can both terminate at near each other on the same lateral side of the support structure 1618. In another more particular embodiment, the ends 1604 and 1606 can terminate at the support structure 1618 spaced apart by a distance of less than 50 cm, or less than 40 cm, or less than 30 cm, or less than 20 cm, or less than 10 cm, or less than 1 cm.

As illustrated in FIG. 16, the first engagement feature 1602 can include a first portion 1610 and a second portion 1612 as seen from a rear view. The second engagement feature 1608 can include a first portion 1614 and a second portion 1616 as seen from a rear view. By way of a non-limiting example and as illustrated in FIG. 16, the first portion 1610 can overlie the first portion 1614, the second portion 1616 can overlie the first portion 1610, and the second portion 1612 can overlie the second portion 1616. In such a manner, the first engagement feature 1602 and second engagement feature 1608 can be woven together such that the first and second engagement features 1602 and 1608 do not come undone when the garment 1600 is removed from the body.

In an embodiment, at least one of the first and second portions 1610 and 1612 or 1614 and 1616 of the first or second engagement features 1602 or 1608, respectively, can have a unitary construction. In another embodiment, the first and second portions 1610 and 1612 or 1614 and 1616 can include different materials as compared to one another. In a particular embodiment at least one of the first or second portions 1610, 1614, 1612, or 1616 can include a first region having a first material property (e.g., elasticity, size, weight) and a second region having a second material property (e.g., elasticity, size, weight) with a different value than the first material property. In an embodiment, a ratio of the first material property to the second material property [first material property/second material property] can be at least 1.01, at least 1.05, at least 1.1, at least 1.2, at least 1.3, at least 1.4, at least 1.5, at least 1.6, at least 1.7, at least 1.8, at least 1.9, at least 2.0, at least 2.5, or at least 5.0. In another embodiment, the ratio [first material property/second material property] can be no greater than 100.0, no greater than 75.0, no greater than 50.0, or no greater than 10.0.

In the past, it was believed that the introduction of too much downward force in the back a garment, such as a bra, capable of providing shoulder alignment improvement would cause the garment to travel upward, thus making the garment less secure. It has been discovered in accordance with embodiments described herein that suitable application of force is achievable without resulting in upward travel of the garment. That is, forces better align using garments in accordance with one or more embodiments described herein. Thus, one can achieve better posture without compromising garment security as was previously believed.

Discrete components are described above with respect to elements like the support structure, arm engagement portions, and engagement features. While the illustrated embodiments include edge lines which visually identify the components, in certain embodiments visual identification between components may not exist. That is, in a particular instance, certain components (e.g., the arm engagement portion and engagement feature) can appear to be a single component instead of a plurality of components coupled together. Moreover, in a particular embodiment, the edges of certain components may extend over or onto other components or replace portions of other components. By way of non-limiting example, at least part of the end 1208 of arm engagement portion 1204 illustrated in FIG. 12 can be replaced or overlapped by an upwardly extending portion of the portion 1212 of the support structure 1214.

Many different aspects and embodiments are possible. Some of those aspects and embodiments are described below. After reading this specification, skilled artisans will appreciate that those aspects and embodiments are only illustrative and do not limit the scope of the present invention. Embodiments may be in accordance with any one or more of the embodiments as listed below.

Embodiment 1. A bra comprising:

a first arm engagement portion adapted to extend around a first shoulder of a wearer on a first lateral side of a wearer's body;

a first engagement feature coupled to the first arm engagement portion and adapted to extend behind a wearer's back toward a second lateral side of the wearer's body;

a support structure coupled to the first engagement feature on the second lateral side of the wearer's body;

a second engagement feature coupled to the support structure on the first lateral side of the wearer's body and adapted to extend behind the wearer's back toward the second lateral side of the wearer's body; and

a second arm engagement portion coupled to the second engagement feature, wherein the second arm engagement portion is adapted to extend around a second shoulder of the wearer on the second lateral side of the wearer's body,

wherein the support structure is adapted to support the wearer's breasts, wherein at least one of the first and second engagement features has an adjustable length, and wherein the first and second engagement features are adapted to float relative to one another.

Embodiment 2. The bra of embodiment 1, wherein the first engagement feature comprises an adjustment element comprising a pull tab.

Embodiment 3. The bra of embodiment 1, wherein at least a portion of the first engagement feature comprises a generally inelastic material.

Embodiment 4. The bra of embodiment 1, wherein the first arm engagement portion has an adjustable length.

Embodiment 5. The bra of embodiment 1, wherein at least part of the first arm engagement portion is disposed within a portion of the support structure.

Embodiment 6. The bra of embodiment 1, wherein at least 25% of a circumferential length of the first arm engagement feature comprises a generally inelastic material.

Embodiment 7. The bra of embodiment 1, wherein the first and second engagement features lie along best fit lines, as viewed in-use, and wherein the best fit lines of the first and second engagement features are adapted to intersect one another at a location adjacent to the wearer's back.

Embodiment 8. The bra of embodiment 1, wherein the first engagement feature is coupled to the support structure at a first location, as viewed in-use, wherein the first engagement feature is coupled to the first arm engagement portion at a location, as viewed in-use, and wherein the first location is at a lower vertical elevation as compared to the second location.

Embodiment 9. The bra of embodiment 1, wherein, as viewed in-use, a vertical line extending along the wearer's back intersects only the first and second engagement features.

Embodiment 10. The bra of embodiment 1, wherein an edge of the support structure extending between the first engagement feature and the second arm engagement portion has a generally arcuate shape.

Embodiment 11. The bra of embodiment 1, wherein, as viewed in-use, the first arm engagement portion has a generally tear-drop shape comprising an apex, and wherein the first engagement feature is coupled to the first arm engagement portion at a location at or adjacent to the apex.

Embodiment 12. The bra of embodiment 1, wherein:

the first engagement feature comprises a first disconnectable element comprising a first part and a second part,

the second engagement feature comprises a second disconnectable element comprising a third part and a fourth part,

in a first configuration, the first and second parts are coupled together and the third and fourth parts are coupled together, and

in a second configuration, the first and third parts are coupled together and the second and fourth parts are coupled together.

Embodiment 13. The bra of embodiment 1, wherein the first arm engagement portion comprises a multi-piece construction comprising at least two discrete components coupled together.

Embodiment 14. A bra comprising:

a support structure adapted to support breasts of a wearer;

a first engagement feature coupled to an outer edge of the support structure on a first lateral side of the wearer and adapted to extend to a shoulder on a second lateral side of the wearer;

a second engagement feature coupled to an outer edge of the support structure on a second lateral side of the wearer and adapted to extend to a shoulder on a first lateral side of the wearer,

wherein, as viewed in-use, a vertical line extending along a back of the wearer intersects only the first and second engagement features, and wherein the first and second engagement features lie along best fit lines that intersect one another.

Embodiment 15. The bra of embodiment 14, wherein the bra is adapted to provide a rearward force to the shoulders on the first and second lateral sides of the body.

Embodiment 16. The bra of embodiment 14, wherein the support structure comprises a band disposed at a lowermost vertical elevation of the support structure, wherein the band is coupled with the first and second engagement features, and wherein the band comprises a material with a lower modulus of elasticity as compared to the first and second engagement features.

Embodiment 17. The bra of embodiment 16, wherein the support structure further comprises cups, and wherein the band is contiguous with the cups along at least 1% of the circumference of the bra.

Embodiment 18. The bra of embodiment 14, wherein the bra comprises an aggregate length of the first engagement feature, second engagement feature, and support structure, as measured by a shortest continuous length thereof, and wherein the aggregate length is adjustable.

Embodiment 19. A method of putting a bra on a wearer's body comprising:

providing a bra having a first engagement feature and a second engagement feature, wherein the first and second engagement features are adapted to extend behind a back of the wearer's body, wherein the first and second engagement features float relative to one another, and wherein the first and second engagement features each lie along best fit lines that intersect one another;

translating the bra over shoulders of the wearer's body; and

adjusting a length of at least one of the first and second engagement features.

Embodiment 20. The method of embodiment 19, wherein the bra further comprises a first arm engagement portion coupled with the first engagement feature and adapted to extend around a shoulder of the wearer's body, and wherein the method further comprises adjusting a length of the first arm engagement portion.

Note that not all of the features described above are required, that a portion of a specific feature may not be required, and that one or more features may be provided in addition to those described. Still further, the order in which features are described is not necessarily the order in which the features are installed or used.

Certain features are, for clarity, described herein in the context of separate embodiments, may also be provided in combination in a single embodiment. Conversely, various features that are, for brevity, described in the context of a single embodiment, may also be provided separately or in any subcombinations.

Benefits, other advantages, and solutions to problems have been described above with regard to specific embodiments, However, the benefits, advantages, solutions to problems, and any feature(s) that may cause any benefit, advantage, or solution to occur or become more pronounced are not to be construed as a critical, required, or essential feature of any or all the claims.

The specification and illustrations of the embodiments described herein are intended to provide a general understanding of the structure of the various embodiments. The specification and illustrations are not intended to serve as an exhaustive and comprehensive description of all of the elements and features of apparatus and systems that use the structures or methods described herein. Separate embodiments may also be provided in combination in a single embodiment, and conversely, various features that are, for brevity, described in the context of a single embodiment, may also be provided separately or in any subcombination. Further, reference to values, for example stated in ranges or lists, includes each and every value within that range or the terminal, end values of the list of values. Many other embodiments may be apparent to skilled artisans only after reading this specification. Other embodiments may be used and derived from the disclosure, such that a structural substitution, logical substitution, or any change may be made without departing from the scope of the disclosure. Accordingly, the disclosure is to be regarded as illustrative rather than restrictive.

1. A bra comprising:
a first arm engagement portion adapted to extend around a first shoulder of a wearer on a first lateral side of a wearer's body;
a first engagement feature coupled to the first arm engagement portion and adapted to extend behind a wearer's back toward a second lateral side of the wearer's body;
a support structure coupled to the first engagement feature on the second lateral side of the wearer's body;
a second engagement feature coupled to the support structure on the first lateral side of the wearer's body and adapted to extend behind the wearer's back toward the second lateral side of the wearer's body; and
a second arm engagement portion coupled to the second engagement feature, wherein the second arm engagement portion is adapted to extend around a second shoulder of the wearer on the second lateral side of the wearer's body,
wherein the support structure is adapted to support the wearer's breasts, wherein the first and second engagement features have adjustable lengths, and wherein the first and second engagement features are adapted to float relative to one another.

2. The bra of claim 1, wherein the first engagement feature comprises an adjustable element comprising a pull tab.

3. The bra of claim 1, wherein at least a portion of the first engagement feature comprises an elastic material.

4. The bra of claim 1, wherein the first and second engagement features are discrete from one another.

5. The bra of claim 1, wherein at least part of the first arm engagement portion is disposed within and statistically coupled to a portion of the support structure.

6. The bra of claim 1, wherein at least 25% of a circumferential length of the first arm engagement portion comprises a generally inelastic material.

7. The bra of claim 1, wherein the first and second engagement features lie along best fit lines, as viewed in-use, and wherein the best fit lines of the first and second engagement features intersect one another.

8. The bra of claim 1, wherein the first engagement feature is coupled to the support structure at a first location, as viewed in-use, wherein the first engagement feature is coupled to the first arm engagement portion at a second location, as viewed in-use, and wherein the first location is at a lower vertical elevation than the second location.

9. The bra of claim 1, wherein, as viewed in-use, a vertical line extending along the wearer's back intersects only the first and second engagement features.

10. The bra of claim 1, wherein an edge of the support structure extending between the first engagement feature and the second arm engagement portion has a concave shape.

11. The bra of claim 1, wherein, as viewed in-use, the first arm engagement portion has a generally tear-drop shape comprising an apex, and wherein the first engagement feature is coupled to the first arm engagement portion at a location at or adjacent to the apex.

12. The bra of claim 1, wherein:
the first engagement feature comprises a first disconnectable element comprising a first part and a second part,
the second engagement feature comprises a second disconnectable element comprising a third part and a fourth part,
in a first configuration, the first and second parts are coupled together and the third and fourth parts are coupled together, and
in a second configuration, the first and third parts are coupled together and the second and fourth parts are coupled together.

13. The bra of claim 1, wherein the first arm engagement portion comprises a first region having a first modulus of elasticity and a second region having a second modulus of elasticity less than the first modulus of elasticity, wherein the first modulus of elasticity is higher than the second modulus of elasticity, and wherein the first region is longer than the second region.

14. A bra comprising:
a support structure adapted to support breasts of a wearer;
a first engagement feature coupled to an edge of the support structure on a first lateral side of the wearer and adapted to extend to a shoulder on a second lateral side of the wearer;
a second engagement feature coupled to an edge of the support structure on a second lateral side of the wearer and adapted to extend to a shoulder on a first lateral side of the wearer,
wherein, as viewed in-use, a vertical line extending along a back of the wearer between the first and second lateral sides of the wearer intersects only the first and second engagement features, wherein the first and second engagement features lie along best fit lines that intersect one another.

15. The bra of claim 14, wherein the first engagement feature has an adjustment element adapted to adjust a length of the first engagement feature, and wherein the adjustment element comprises a pull tab.

16. The bra of claim 14, wherein the support structure comprises a band disposed at a lowermost vertical elevation of the support structure, wherein the band is coupled with the first and second engagement features, and wherein the band comprises a material with a lower modulus of elasticity as compared to the first and second engagement features.

17. The bra of claim 14, wherein the first and second engagement features are adapted to be adjusted by adjustable elements disposed between the first and second lateral sides of the back of the wearer.

18. The bra of claim 14, wherein the first engagement feature is coupled with an arm engagement portion adapted to be disposed around a shoulder of the wearer on the second lateral side of the wearer's body, and wherein at least 25% of the circumference of the arm engagement portion comprises a generally inelastic material.

19. A method of putting a bra on a wearer's body comprising:
providing a bra having a first engagement feature and a second engagement feature, wherein the first and second engagement features are adapted to extend behind a back of the wearer's body, wherein the first and second engagement features float relative to one another, and wherein the first and second engagement features each lie along best fit lines that intersect one another;
translating the bra over shoulders of the wearer's body; and
adjusting a force on at least one of the shoulders of the wearer by adjusting an operable length of the first or second engagement features.

20. The method of claim 19, wherein adjusting force on the shoulders is performed by:
pulling on a pull tab coupled to and adapted to adjust the first engagement feature, and
pulling on a second pull tab coupled to and adapted to adjust the second engagement feature,
wherein the first and second pull tabs are disposed along the back of the wearer.
